(19) United States
(12) Patent Application Publication
(10) Pub. No.: US 2023/0115785 A1
(43) Pub. Date: Apr. 13, 2023

(54) ANALYTE DETECTION

(71) Applicant: University of Leeds, Leeds (GB)

(72) Inventors: Mukhil Raveendran, Freiburg im Breisgau (DE); Paolo Actis, Leeds (GB); Christopher Paul Walti, Leeds (GB); Andrew James Lee, Leeds (GB)

(21) Appl. No.: 17/801,759

(22) PCT Filed: Feb. 26, 2021
(86) PCT No.: PCT/GB2021/050503

§ 371 Date: Aug. 23, 2022

(30) Foreign Application Priority Data
Feb. 28, 2020 (GB) 2002891.6

(51) Int. Cl. G01N 33/52; C12Q 1/6816; B82Y 5/00; G01N 33/487

(52) U.S. Cl. CPC G01N 33/52; C12Q 1/6816; B82Y 5/00; G01N 33/48721; C12Q 2565/631

(57) ABSTRACT

A carrier molecule for the detection of a target analyte comprises a molecular frame which defines a central void, and a binding moiety which is specific for the target analyte. The binding moiety is bound to the frame and positioned such that the target analyte, when bound to the binding moiety, is located in the central void. The carrier molecule finds use in the detection and/or quantification of target analytes in a sample.

The present invention relates to a carrier molecule for the detection of a target analyte, a composition and a kit comprising a carrier molecule, and a method of detecting a target analyte in a sample using a carrier molecule. More particularly, the invention relates to a carrier molecule comprising a molecular frame to which is bound a binding moiety that is specific for the target analyte.

Rapid and low-cost detection of disease biomarkers is becoming increasingly important in modern healthcare where a growing focus is placed on early diagnosis. This presents a considerable technological challenge as the relevant biomarkers are often only present at very small concentrations. Ideally, a diagnostic assay should be able to detect the presence of very few biomarkers in a small volume of a complex clinical sample. The current gold-standard clinical diagnostic assays are generally based on ensemble-averaging immunoassays such as ELISAs (enzyme-linked immunosorbent assay). In these, a binding moiety such as an antibody or antibody mimetic is used to capture relevant biomarkers in the sample and, in general, each antibody-biomarker interaction contributes a small amount to the accumulated assay signal. However, the individual immuno-interactions cannot be identified anymore and only manifest themselves as of this ensemble-averaged signal. Arguably, the ability to detect biomarkers with single entity resolution rather than via ensemble averaging techniques provides significant advantages for the detection of ultra-small biomarker concentrations.

Of the many single molecule methods which have been developed over recent years, the use of nanopores, where a voltage is applied across the nanopore and pulses in the time-varying electrochemical current, for detecting individual proteins is a promising approach. Here, single proteins which translocate through the nanopore cause a momentary modulation in the otherwise steady ion current. This approach has been used in a number of single molecule protein and DNA detection studies.

However, despite these advances, there remain significant challenges. It is very difficult to detect a specific molecule from a complex mixture as the translocation speed of single entities is generally high when the diameter of the nanopore is larger than the size of the molecule, and the translocation therefore results in only a weak signal, and correspondingly low signal-to-noise. Furthermore, the signals resulting from different proteins are generally very similar and difficult to differentiate. To address these limitations, a variety of strategies have been implemented, including to slow down protein translocations through careful selection of electrolytes and the development of high bandwidth electronics. Also, chemical and biological modification of nanopore and use of hybrid nanopores have been explored to increase sensitivity and selectivity. More recently, large carrier molecules including nanoparticles, antibodies, and long linear dsDNA molecules have been exploited to host the subject molecule and thereby increase the mass and consequently reduce the translocation speed of the subject molecule.

While DNA-based carriers have been used successfully and provide a suitable carrier system owing to the ease of modifying and engineering the system, the long linear DNA carriers are not without limitations—they are prone to forming knots and kinks which are known to provide false positive signals during nanopore translocation. Furthermore, significant variations in the signal have been noted, depending on the translocation orientation and high blockage rates due to the passage of multiple DNA molecules through the nanopore. In an attempt to circumvent these limitations, recent work has combined single molecule fluorescence with nanopore detection to perform single molecule binding assays. This approach, although elegant, negates the key advantage of nanopore sensing by introducing labelling and complex optical setups as opposed to a simple electrical read-out system which does not require labelling.

The present invention has been devised with these issues in mind.

According to a first aspect of the invention, there is provided a carrier molecule for the detection and/or quantification of a target analyte, the carrier molecule comprising:
a molecular frame which defines a central void; and
a binding moiety which is specific for the target analyte, wherein the binding moiety is bound to the frame and positioned such that the target analyte, when bound to the binding moiety, is located in the central void.

The molecular frame may be formed from nucleic acid, protein, peptide, or a mixture thereof.

Thus, in some embodiments, the molecular frame comprises a nucleic acid. The nucleic acid may be DNA, RNA, or a nucleic acid analogue, or a mixture thereof.

As used herein, a ‘nucleic acid analogue’ is understood to mean a structural analogue of DNA or RNA, designed to hybridise to complementary nucleic acid sequences. A nucleic acid analogue may be distinguished from DNA or RNA by its phosphate backbone, sugar groups, and/or nucleobases. Examples of nucleic acid analogues include, but are not limited to, threose nucleic acid, glycol nucleic acid, morpholino oligomers, peptide nucleic acids (PNA), locked nucleic acids “LNA”, 2′-O-methyl nucleic acids, 2′-fluoro nucleic acids, phosphorothioates, and metal phosphonates.

In some embodiments, the molecular frame is formed partially or entirely from nucleic acid. In other words, the molecular frame may be a nucleic acid frame.

In some embodiments, the nucleic acid is DNA.

A nucleic acid frame may be formed by DNA origami (or an equivalent process which uses RNA or nucleic acid analogues instead or in addition to DNA). As is known in the art, DNA origami is the nanoscale folding of DNA to create non-arbitrary two- or three-dimensional shapes at the nanoscale. Generally, the process involves the folding of one or more long “scaffold” strands of DNA into a particular shape using a plurality of rationally designed “staple” DNA strands. Each staple is designed such that it hybridises to a specific region of the scaffold and thus via base-pairing the scaffold strands are formed into a desired shape. Methods for the formation of DNA origami structures are described herein, as well as, for example, by Rothemund. Nature 440: 297-302 (2006), Douglas et al., Nature 459:414-418 (2009) and Dietz et al., Science 325: 725-730 (2009). Software for the design of staples required to form a particular shape is available, e.g. caDNAno, which is an open source software for creating origami structures from DNA.

Thus, in some embodiments the nucleic acid frame may be a DNA origami structure. DNA origami structures have been shown to substantially resist degradation by DNAses present in biological fluids. Methods for increasing the stability of DNA origami structures, for example using cross linking, will be known to the skilled person. Accordingly, the concentration and longevity of DNA origami structures in biological samples provides a more reliable means of detecting and/or quantifying target analytes.

The molecular frame may be formed from at least one scaffold strand and a plurality of staple strands. In some embodiments, the scaffold strand(s) may be derived from a bacteriophage, such as the bacteriophage M13, e.g. M13mp18.

In some embodiments, the molecular frame is formed, in part or entirely, from one or more peptides and/or proteins. In other words, the molecular frame may be a peptide or protein frame, or a nucleic acid-peptide hybrid frame. Methods of creating protein origami structures are described by Ljubetic et al., Nat. Biotechnol., 35, 1094-1101 and Lapenta et al., Chem, Soc. Rev. 47, 3530-3542.

As used herein, the term “frame” will be understood to be a continuous structure of a defined shape. In other words, the frame is a thin, sheet- or tile-like structure, having an aperture therethrough.

The advantage of using a molecular frame as a carrier molecule, as opposed to linear DNA carriers, is that linear carriers can form kinks and knots, causing false positive signals when they pass through a nanopore. A structured carrier molecule like the molecular frame of the invention, reduces or avoids this problem.

The frame may therefore be considered to comprise a first (e.g. upper) surface and a second, opposing (e.g. lower) surface. Thus, both the first and second surfaces may be described as external surfaces. Both surfaces have an aperture therein. Therefore, in some embodiments, the frame does not comprise an inner (or internal) surface.

WO2012/061719 describes DNA origami devices for use in targeted drug delivery. The DNA origami devices are able to sequester potentially biologically active moieties within the interior of the device, thereby sterically preventing them from interacting with inappropriate cell populations. The DNA origami device may be barrel-shaped, e.g. a hexagonal tube.

Unlike the DNA origami device of WO2012/061719, the carrier molecule of the present invention, and the frame thereof, is not able to sequester molecules (such as biologically active molecules) such that they are prevented from interacting with other molecules or cells.

The frame of the present invention is not a container. In other words, the frame does not completely enclose analytes within the frame. Unlike the DNA origami device of WO2012/061719, the carrier molecule of the present invention binds the target analyte in a void which is defined by a thin frame. A target analyte which is bound by the binding moiety and located in the central void of the frame may therefore protrude from the void and extend beyond the first and/or second surface of the frame. Thus, the frame does not preclude a target analyte, which is bound by the binding moiety and located in the central void of the frame, from interacting with the environment, or molecules or cells therein.

In some embodiments, the molecular frame of the invention has a single conformation. In other words, unlike the DNA origami device of WO2012/061719, it may be that the molecular frame is not substantially able to change shape, such as between an open conformation and a closed conformation. This means that at least a portion of a target analyte, when bound by the binding moiety, is exposed to the surrounding environment, including any molecules or cells therein, at all times. At least a portion of the target analyte may extend outside of the frame. Thus, the molecular frame may be configured such that at least a portion of a target analyte, when bound by the binding moiety, extends outside of the frame. The target analyte may extend outside of the frame in one dimension only. For example, the target analyte may be contained within the central void in a first and a second dimension (e.g. in the directions of the length and height of the frame), but extend beyond the boundaries of the frame in a third dimension (e.g. in the direction of the thickness of the frame).

The molecular frame may be of any suitable shape. For example, the frame may be rectangular, square, circular, oval, triangular, trapezoid, rhomboid, pentagonal, hexagonal, octagonal, kite-shaped or irregular in shape. The frame may comprise three, four, five or more sides (or “arms”). The sides of the frame may be all of the same length (e.g. in embodiments wherein the frame has the shape of a square or an equilateral triangle), or some or all of the sides may be of different lengths (e.g. in embodiments wherein the frame is rectangular).

In some embodiments, the molecular frame is rectangular or square. Without being bound by theory, it is thought that a rectangular or square frame may enhance detection as the carrier molecule translocates through a nanopore, since a rectangular or square frame is better able to align with the electric field lines.

The molecular frame of the present invention may be substantially 2-dimensional. It will be appreciated by the skilled person that, in this field of art, “2-dimensional” (or “2D”) is understood as meaning that the frame is significantly larger in two of its dimensions than in the third dimension (and thus is more accurately described using 2D geometric shapes), rather than meaning that the frame is a literal 2-dimensional object. Thus, in some embodiments the molecular frame is not tube shaped.

In some embodiments, the frame has a length, a height and a thickness. The thickness of the frame may be small relative to the length and/or the height of the frame. It will be understood that “thickness” as used herein refers to a dimension of the frame as measured at a single point on a side of the frame. The thickness of the frame is the distance from the first (upper) surface to the second, opposing (lower) surface of the frame.

The thickness of the frame may be no more than about 50 nm, no more than 20 nm, no more than 15 nm, no more than about 10 nm, no more than about 8 nm, no more than about 6 nm, no more than about 4 nm or no more than about 3 nm.

It will be appreciated that, when used in the context of the molecular frame as a whole, the terms “length” and “height” refer to the external dimensions of the frame. For example, the length may be measured from a point on the outer edge of frame to a directly opposing point on the outer edge of the frame. It will also be appreciated that the terms “length” and “height” refer to the maximum dimensions of the frame, in any direction.

In some embodiments, the ratio of the length and/or height of the frame to the thickness of the frame is at least 5:1, at least 10:1, at least 15:1, at least 20:1 or at least 50:1. It may be that the ratio of the length and/or height of the frame to the thickness of the frame is from 5:1 to 100:1, from 8:1 to 70:1, from 10:1 to 50:1 or from 15:1 to 30:1.

For example, the length of the frame may be at least 30, at least 50, at least 70, at least 80, at least 90, at least 100 nm, at least 150 nm or at least 200 nm.

The height of the frame may be at least 30, at least 50, at least 70, at least 80, at least 90, at least 100 nm, at least 150 nm or at least 200 nm.

In some embodiments (e.g. in embodiments wherein the frame has the shape of a square), the length of the frame may be substantially the same as the height of the frame.

The carrier molecule may be a nanostructure. The largest (external) dimension of the frame may be no more than 1000 nm, no more than 800 nm, no more than 500 nm, no more than 300 nm, no more than 200 nm, no more than 100 nm or no more than 50 nm.

In some embodiments, the molecular frame is substantially planar (i.e. substantially flat).

In other embodiments, the molecular frame is curved. In such embodiments, the frame may have a concave first surface and a convex second surface. The frame may be curved to a limited extent. For example, the frame will not be curved to the extent that opposing edges of the frame meet to form a cylindrical or tube-like structure.

In some embodiments, the curve of the frame is such that the frame has an overall depth which is no more than 50%, no more than 30%, no more than 20%, no more than 10% or no more than 5% of the height and/or length of the frame. It will be appreciated that “depth” in this context refers to the overall depth of the frame as a whole (i.e. the global depth), as opposed to the thickness of the frame which refers to the (local) dimension of a given point on a side of the frame. In embodiments in which the frame is curved, the depth of the frame may be greater than, or significantly greater than, the point thickness of the frame.

The depth of the frame may be no more than 200 nm, no more than 150 nm, no more than 100 nm, no more than 80 nm, no more than 60 nm, no more than 50 nm, no more than 40 nm, no more than 30 nm, no more than 20 nm, no more than 10 nm, no more than 6 nm, or no more than 5 nm.

It has been found by the present inventors that a molecular frame, such as a nucleic acid frame, is particularly suited to the detection of a target analyte by translocation through a nanopore, for example using a nanopipette. This is because the presence of a void or cavity in the centre of the molecular frame causes a splitting of the peak in the ion current signature generated as the molecular frame translocates through a nanopore, thereby producing a double peak. Surprisingly, the inventors have now found that by providing a molecular frame with a binding moiety in or proximal to the central void to which the target analyte can bind specifically, the presence or absence of the analyte in the central void can be detected using a nanopore by observing the ion current signature, which is characteristic of the carrier molecule. In particular, the inventors have found that the binding of a target analyte to the binding moiety, which may be positioned in or close to the central void of the molecular frame, causes a change in the peak of the ion current signature from a double peak (for an unoccupied carrier molecule) to a single peak (for an occupied carrier molecule, i.e. having a target analyte bound in the central void), due to the target analyte partially filling the void. In other words, the frame structure enables analyte detection by detecting occlusion of the void in the frame.

The relationship between the observed translocation ion current signature and the void geometry within the nucleic acid frame thereby enables the detection of much smaller analytes using nanopores.

It may be that the carrier molecule is for detecting a target analyte using a nanopore. Thus, the carrier molecule of the invention may be suitable for carrying (i.e. transporting, or translocating) a target analyte through a nanopore. The carrier molecule may not, itself, be a nanopore. It will therefore be appreciated that, in use, the carrier molecule may be unbound (other than to the target analyte, if present). For example, the carrier molecule may exist freely in solution.

In some embodiments, the carrier molecule is configured such that, in the absence of a bound target analyte, the carrier molecule produces a double peak in an ion current signature upon translocation through a nanopore. The carrier molecule may be configured such that when a target analyte is bound by the binding moiety, a single peak in the ion current signature is generated upon translocation of the carrier molecule through a nanopore. Thus, the carrier molecule may be configured such that, in use, binding of the target analyte to the carrier molecule modulates the signal from a double to a single peak. This type of signal modulation is particularly advantageous because it provides clear evidence of a bound analyte, as opposed to, for example, relying on detecting a change in the width of a signal peak to determine the presence of a bound analyte.

Without being bound by theory, it is believed that a change in the signal from a double peak to a single peak upon analyte binding may be due to the thin frame-like structure of the carrier molecule, for example the substantially 2D structure. It is thought that if a tube-like structure (i.e. a 3D structure) is used as a carrier molecule, the presence of the internal void may not be detected during translocation of the carrier molecule through a nanopore. As the thickness of the carrier molecule increases, e.g. from substantially 2D to 3D, the ability to detect the presence of the internal void is believed to be lost. Thus, the use of a thin molecular frame, for example a substantially 2D structure, advantageously enables a change in signal to be readily observed upon binding of the target analyte.

Advantageously, the molecular frame of the carrier molecule has a stable and semi-rigid structure. This overcomes the problems commonly associated with the use of long, linear DNA carriers, such as the DNA passing through the pore in a non-linear or folded arrangement, which can make it more difficult to distinguish individual analytes. In some embodiments, the molecular frame is substantially rigid, or non-deformable. A substantially rigid structure may be beneficial for improving the nanopore signal.

The frame may be formed from one or more layers of a nucleic acid, peptide, or protein. For example, the frame may be formed of one, two, three, four or more layers of nucleic acid, peptide, or protein.

In some embodiments, the frame is formed from a single layer of a nucleic acid, peptide or protein.

The frame may be formed entirely or partially from a double-stranded nucleic acid, e.g. double stranded DNA. For example, while the majority of the frame may be formed from a double-stranded nucleic acid, the frame may also comprise portions of single-stranded nucleic acid. Double-stranded nucleic acid is beneficial for providing stability to the frame.

In some embodiments, the thickness of the frame corresponds to the diameter of one double-stranded nucleic acid helix, e.g. 2-3 nm.

In some embodiments, the frame is formed from two layers of nucleic acid e.g. two layers of double-stranded nucleic acid. In such embodiments the frame may have a thickness of from about 4 nm to about 8 nm, or from about 5 nm to about 7 nm.

Advantageously, the size of the molecular frame, and/or the size of the central void therein, can be tailored according to the size of the target analyte. Thus, the central void may be sized such that it is capable of receiving the analyte therein. In particular, the size of the void may be selected such that upon binding of the analyte within the central void (via the binding moiety), the ion current signature of the carrier molecule changes from a double peak to a single peak.

The carrier molecule may be configured such that the target analyte, upon binding to the binding moiety, substantially fills or occludes the void. Substantial occlusion of the void by a bound target analyte may be achieved by tailoring one or more of: the dimensions of the molecular frame; the dimensions of central void; and the dimensions and/or location(s) of the binding moiety or moieties. The skilled person will be capable of selecting the features of the carrier molecule so as to achieve substantial occlusion of the void by a target analyte, depending on the target analyte of interest. Capture of the target analyte within the frame is advantageous since it ensures a stronger signal, and helps to avoid entanglement.

The molecular frame may also be sized such that it can pass through a nanopore. In some embodiments the molecular frame (or carrier molecule) is sized so as to allow the passage of a single molecular frame (or carrier molecule) through the nanopore at a time.

In some embodiments, the central void may have a length of at least 10, at least 20, at least 30, at least 50 nm or at least 100 nm.

The central void may have a height of at least 10, at least 20, at least 30, or at least 50 nm. It will be appreciated that in the context of the central void, “length” and “height” refer to the internal dimensions of the frame, i.e. as measured from a point on an inner edge of frame to a directly opposing point on an inner edge of the frame. The central void may have an area which is the multiple of the height and the length of the void.

In some embodiments the carrier molecule is configured such that the target analyte, upon binding to the binding moiety, occupies at least 50%, at least 60%, at least 70%, at least 75%, at least 80%, at least 85%, at least 90%, or at least 95% of the area of the central void. In some embodiments the target analyte, when bound by the binding moiety and located in the central void, substantially fills or occludes the void.

The dimensions of the central void may be defined with reference to the width of the individual sides (i.e. arms) of the frame. The width of each side is defined as the distance from a point on the inner edge of a side (i.e. proximal to the void) to an opposing point on the outer edge (i.e. distal to the void) of the same side. For example, in a square frame, the central void may be a square defined by four sides of equal length and height. The length and height of the void may be the same as the width of each side of the frame, Thus, the ratio of the height/length of the void to the width of each side is 1:1.

In a further example, the frame is rectangular and defines a rectangular void. The void may have a length which is twice the height of the void. The ratio of the height of the void to the width of each side may be 1:1. The ratio of the length of the void to the width of each side may therefore be 2:1.

Thus, in some embodiments, the ratio of the height and/or length of the central void:the width of a for each) side of the frame is from 0.5:1 to 5:1, from 1:1 to 3:1 or from 2:1 to 2.5:1.

In some embodiments, the ratio of the surface area of the void:the surface area of the frame is from 1:1 to 25:1, from 1:2 to 15:1, from 1:3 to 10:1 or from 1:5 to 1:8.

The central void may be the same shape as the molecular frame, or it may be a different shape. In some embodiments, the central void has the same general shape as the frame (e.g. both may be rectangular), but the relative dimensions of the central void may differ to those of the frame, such that the central void and the frame are asymmetric.

The dimensions of the nucleic acid frame may be confirmed using microscopy, e.g. AFM.

The desired dimensions of the frame and the central void can be achieved by tailoring the scaffold and staple strands, as is known in the art.

The binding moiety may comprise any suitable molecule which is able to selectively bind to the target analyte. Examples of such molecules include aptamers (nucleic acid or peptide aptamers), affimers, antibodies (or derivatives or fragments thereof such as domain antibodies, single chain variable region fragments, humanised antibodies, bi-specific antibodies, Fab and F(ab)₂ fragments), proteins, molecularly imprinted polymers (MIPs) and nucleic acid-protein fusion molecules.

In some embodiments the binding moiety comprises an aptamer (e.g. a DNA aptamer).

In some embodiments, the carrier molecule comprises at least two, at least three or at least four binding moieties. In some embodiments, at least two, at least three or at least four binding moieties may be bound to a single frame. In such embodiments it will be appreciated that the binding moieties will all be specific for the same target analyte.

Alternatively, the carrier molecule may comprise a single binding moiety.

The binding moiety is bound to the frame. The binding moiety may be configured (e.g. sized and/or positioned) such that the target analyte, when bound to the binding moiety, is located in the central void. In some embodiments, the binding moiety may be configured such that the target analyte, when bound to the binding moiety, substantially fills (i.e. occludes) the central void.

In some embodiments, the binding moiety is located in the central void.

In some embodiments, the binding moiety is located proximal to the central void (e.g. closer to an inner edge of the frame than an outer edge of the frame).

The binding moiety may be covalently bound to the molecular frame, or it may be non-covalently bound, for example by electrostatic interactions or hydrogen bonding.

In some embodiments the binding moiety is bound to the molecular frame via an anchor moiety.

In some embodiments the anchor moiety is formed from a nucleic acid, e.g. DNA. The anchor moiety may be an extension of a nucleic acid frame. For example, the anchor moiety may constitute a portion of a scaffold or staple strand which forms the nucleic acid frame.

The anchor moiety may comprise a single-stranded nucleic acid sequence which is complementary to a sequence that forms a part of the binding moiety. The binding moiety can therefore be bound to the nucleic acid frame via nucleic acid (e.g. DNA) hybridization.

Thus, in some embodiments the binding moiety comprises a single-stranded nucleic acid sequence for hybridising to the anchor moiety. For example, an aptamer binding moiety may be modified so that it comprises a single-stranded DNA sequence at its 3′ or 5′ end.

The anchor moiety must be located such that the target analyte (when bound to the binding moiety), is positioned inside the frame, i.e. in the central void. This is so that upon binding of the target analyte, the ion peak signature is changed from a double to a single peak. In some embodiments, the anchor moiety is located in the central void of the molecular frame.

Similarly, the binding moiety may have a length which is selected such that, when a target analyte is bound to the binding moiety, the target analyte is located in the central void of the molecular frame. For example, the binding moiety may be of a length suitable to bind the target analyte in such a way that the target analyte substantially occludes the central void of the frame. Additionally, or alternatively, the binding moiety may have a configuration which locates the target analyte in the central void. For example, the binding moiety may be configured such that upon binding of the target analyte, the folding of the binding moiety is altered so as to locate the target analyte in the central void. The positioning of the target analyte in the central void enables the signal of the carrier molecule, upon passage of the carrier molecule through a nanopore, to be modulated from a double to a single peak by the presence of a bound target analyte.

Advantageously, the carrier molecules of the invention may be provided with barcodes. This enables the carrier molecules to be used for multiplexing e.g. for the detection of multiple different target analytes in a given sample.

A carrier molecule for the detection and/or quantification of a target analyte in a multiplex system may comprise:
a detection region comprising a molecular frame which encloses a central void, and a binding moiety which is specific for the target analyte, wherein the binding moiety is bound to the frame and positioned such that the target analyte, when bound to the binding moiety, is located in the central void; and
a barcode region attached to the detection region.

The molecular frame with a binding moiety bound thereto, which is comprised within the detection region, may also be referred to as a “detection unit” or “detection frame”.

The detection region may comprise a single detection frame, or it may comprise multiple detection frames, for example two, three or more detection frames.

In some embodiments, the detection region comprises a first detection frame to which is bound a first binding moiety, and a second detection frame to which is bound a second binding moiety. The first and second binding moieties may be specific for different target analytes, or they may be specific for the same target analyte.

The barcode region may comprise at least one nanostructure e.g. a nucleic acid, protein or peptide nanostructure. In some embodiments, the barcode region comprises a plurality of nanostructures, e.g. two, three, four, five, six or more nanostructures. The nanostructures may be arranged in series.

The nanostructure may be a molecular tile, or it may be a molecular frame which lacks a binding moiety (such that it cannot bind an analyte). As used herein, a molecular “tile” refers to a structure which does not comprise a central void. In other words, the tile is a solid or “filled” shape.

The barcode region may thus comprise molecular tiles, or molecular frames lacking binding moieties, or a mixture of both. The nanostructure(s) of the barcode region may be referred to as “identifier units”.

Like the molecular frame(s) of the detection region (i.e. the detection frames), some or all of the nanostructures may be substantially planar, or 2D. Alternatively, some or all of the nanostructures may be curved. Some or all of the nanostructures may be of similar size and geometry to the detection frame(s). The nanostructures may be formed by DNA or protein origami.

The detection region may also comprise one or more nanostructures (e.g. one or more molecular tiles and/or frames lacking binding moieties), in addition to the detection frame(s). The nanostructures present in the detection region may be joined in series with the detection frame(s). The nanostructures present in the detection region may serve to space the detection frames from each other, and/or act as orientation markers.

The detection frames and the nanostructures may be joined together in series to form a chain or “ribbon”. The detection frames and the nanostructures may thus be considered to constitute subunits of the chain or ribbon.

The subunits of the chain (i.e. the molecular frames and tiles) may be formed from a single scaffold strand.

Alternatively, the subunits of the chain may be separately formed from individual scaffold strands. The individual subunits may then be joined together.

Adjacent molecular frames and tiles (i.e. subunits) may be joined together by at least one linker moiety. In some embodiments, from 1 to 6 linker moieties are provided between adjacent subunits, e.g. 2, 3, 4 or 5 linker moieties.

In some embodiments the linker moieties comprise nucleic acid sequences, e.g. double-stranded DNA or RNA.

Each subunit (i.e. each detection frame and nanostructure) may comprise a single-stranded nucleic acid sequence which is complementary to a single-stranded nucleic acid sequence on an adjacent subunit. The linker moieties are then formed upon hybridisation of the single-stranded sequences. The single-stranded nucleic acid sequences which form the linkers may protrude from edges of the subunits.

The linker moieties may be from 10 to 50, from 15 to 35 or from 20 to 30 base pairs in length.

Thus, in some embodiments a carrier molecule for the detection of a target analyte in a multiplex system may comprise a plurality of, e.g. two, three, four, five, six or more nanostructures in series, wherein at least one of the nanostructures is a molecular frame (e.g. a nucleic acid frame) functionalised with a binding moiety specific for a target analyte.

The nanostructures enable identification of the carrier molecule, for example by their shape, size and/or by the signal produced as the unit translocates through a nanopore.

It will be appreciated that the molecular frames of the carrier molecule (including the detection frames in the absence of bound analyte and the frames of the barcode region which lack a binding moiety) will produce a double peak as they translocate through a nanopore. In contrast, the molecular tiles of the barcode region will produce a single peak as they translocate through a nanopore. The amplitude and dwell time of the peaks will be determined by the dimensions of the frames and tiles. As such, a characteristic ion current signature can be produced by tailoring the number, dimensions and order of the nucleic acid nanostructures present in the carrier molecule. This enables identification of the carrier molecule by its unique signature.

For example, a carrier molecule may comprise four molecular frames joined in series, wherein the first frame of the series has a binding moiety bound thereto and located in the central void of the frame, while the second, third and fourth frames lack a binding moiety. The first frame of the series thus constitutes the detection region, and the second, third and fourth frames constitute the barcode region. The first frame in the series will capture a target analyte present in a sample, and produce a single peak (sp) signal as it translocates through a nanopore. The second, third and fourth frames, which are unable to bind an analyte in the central void, will generate double peak (dp) signals as they translocate through the nanopore. The carrier molecule as a whole therefore produces the following signal: sp-dp-dp-dp. Since the dwell time and amplitude of the peaks can be varied by modifying the size of the frames and the central void, the geometry of the frames can be tailored to provide a unique “barcode” which is characteristic of a given carrier molecule. Different carrier molecules having different barcodes can be used to detect different target analytes by providing a different binding moiety in each different type of carrier molecule, thereby enabling multiplexing.

While the above example describes a carrier molecule comprising a chain of four molecular frames in series, wherein the frame at the terminus of the chain has a binding moiety bound thereto, it will be appreciated that the number, type and order of nucleic acid nanostructures in the series can be varied as desired.

According to a third aspect of the invention, there is provided a composition comprising a carrier molecule as described herein. The composition may comprise the carrier molecule in a solution, e.g. an aqueous solution.

The carrier molecule of the present invention finds use in the detection and/or quantification of a target analyte. Thus, the invention further provides the use of a carrier molecule for detecting and/or quantifying the presence of a target analyte in a sample. The carrier molecule may be as described herein. Said use may comprise translocating the carrier molecule through a nanopore. A difference in the signal generated by a carrier molecule alone versus a carrier molecule with a bound target analyte enables the presence of the analyte and/or its concentration to be determined.

According to a further aspect of the invention, there is provided a complex comprising a carrier molecule as defined herein, bound to a target analyte.

Thus, the complex may comprise:
a carrier molecule comprising a molecular frame which defines a central void, and a binding moiety which is specific for the target analyte, wherein the binding moiety is bound to the frame; and
a target analyte bound to the binding moiety and located in the central void.

In some embodiments, the carrier molecule is for the detection of a target analyte in a multiplex system, as described herein.

In a further aspect, the invention provides a system for detecting and/or quantifying a target analyte in a sample, the system comprising:
a first electrolyte reservoir and a second electrolyte reservoir, the first and second reservoirs being separated by a barrier comprising a nanopore; and
optionally, electrodes for translocating molecules through the nanopore from the first electrolyte reservoir to the second electrolyte reservoir,
wherein at least one of the first and second electrolyte reservoirs comprises carrier molecules as described herein.

The system may comprise a chamber containing an electrolyte solution, wherein the chamber is separated into first and second electrolyte reservoirs by the barrier.

The system may comprise a pair of electrodes, i.e. an anode and a cathode. The anode may be immersed in one of the first and second electrolyte reservoirs, and the cathode may be immersed in the other of the first and second electrolyte reservoirs.

It may be that the carrier molecules are located in the first electrolyte reservoir, or the second electrolyte reservoir, or in both of the first and second electrolyte reservoirs. The carrier molecules may exist freely in the electrolyte solution (i.e. they are unbound).

For detecting the target analyte using the system, the sample may be added to the first and/or second electrolyte reservoir. It will be appreciated that the sample will be added to a reservoir which comprises carrier molecules. Thus, in some embodiments the system further comprises the sample in the first and/or second electrolyte reservoir.

The nanopore enables passage of molecules between the first and second electrolyte reservoirs. Translocation of the carrier molecules, with bound target analyte if present in the sample, from one of the reservoirs to the other through the nanopore may be driven by an applied voltage between the electrodes, or by pressure.

The barrier may comprise or consist of a membrane, such as a biological membrane (e.g. formed from a lipid or phospholipid bilayer, liposomes or polymer film) or a solid-state membrane (e.g. formed from silicon oxide, silicon nitride, hafnium oxide, graphene or aluminium oxide, or a combination thereof).

The nanopore may be as described herein.

According to a further aspect, the invention provides a method for detecting and/or quantifying the presence of a target analyte in a sample, the method comprising:
contacting a carrier molecule with the sample; and
detecting the presence of a carrier molecule-target analyte complex.

The carrier molecule used in the method may be the carrier molecule as described herein.

The method may further comprise incubating the carrier molecule with the sample for a time sufficient to enable the target analyte to bind to the binding moiety of the carrier molecule, thereby forming carrier molecule-target analyte complex.

The carrier molecule may be incubated with the sample for at least 1, at least 2, at least 5, at least 15, at least 30, at least 45 or at least 60 minutes.

Incubation may be carried out at a temperature of from 10 to 40° C., from 12 to 37° C. or from 15 to 30° C. In some embodiments, incubation is carried out at room temperature, e.g. 19-22° C., or about 20° C.

The presence of the carrier molecule-target analyte complex may be detected by any suitable technique, such as microscopy (e.g. AFM) or gel electrophoresis.

In some embodiments, the carrier molecule-target analyte complex is detected by voltage-driven translocation of the complex through a nanopore.

A change in the ion current signature upon translocation, as compared to the signature of the carrier molecule alone (i.e. in the absence of the target analyte), may be indicative of the formation of a carrier molecule-target analyte complex and thus the presence of the target analyte in the sample.

The change in the ion current signature may be a change in the peak shape, the peak amplitude and/or the dwell time. For example, binding of the analyte to the carrier molecule may cause an increase in the peak amplitude, a decrease in the dwell time, and/or a change in the peak shape from a double to a single peak.

In some embodiments, detecting the presence of a carrier molecule-target analyte complex comprises detecting a change in the peak shape, amplitude and dwell time as the carrier molecule is translocated through a nanopore.

Ideally, the nanopore is sized so as to allow the passage of a single carrier molecule through the pore at a time. It will therefore be appreciated that the size of the nanopore may be selected by the skilled person in accordance with the size of the carrier molecule.

In some embodiments, the nanopore may be at least 5, at least 10, at least 30, at least 50, at least 70, at least 100 or at least 130 nm in diameter. The nanopore may be no more than 200 nm, no more than 150 nm or no more than 100 nm in diameter. It will be appreciated that nanopores are not necessarily circular. As such, the “diameter” of the nanopore in this context refers to the average dimension of the pore. While large pore sizes are not suitable for the detection of single analytes or linear dsDNA carriers, they can be used for larger structures, such as DNA or protein origami structures. Furthermore, by tuning the size of the nanopore to the size of the carrier molecule, such that unbound target and non-target analytes cannot be detected, there is less background signal. As a result, the signal produced requires less filtering during subsequent analysis. In addition, larger nanopores are easier to fabricate reliably.

The present invention thus provides a flexible system which can be tailored for the detection of a desired target analyte by designing a carrier comprising molecular frame having a central void which is sized to receive the target analyte therein, and by selecting a nanopore which is sized so as to allow the passage of a single carrier molecule at a time therethrough.

The nanopore may be a solid state nanopore, or a biological nanopore. A solid state nanopore may be formed in a membrane made from silicon oxide, silicon nitride, hafnium oxide, graphene, aluminium oxide, or combinations thereof.

The nanopore may be located at the tip of a nanopipette. In some embodiments, the nanopipette is a glass nanopipette. Nanopipettes may be fabricated using the methods described herein, or other methods which will be known to those skilled in the art.

As is known in the art, nanopipettes are a class of nanopores which can be used for the detection and analysis of single molecules in solution. Nanopipettes can be easily fabricated with highly controlled pore sizes, making then a cost effective alternative to traditional solid state nanopores. The detection and analysis of a single molecule with nanopipettes relies on resistive pulse sensing. For this, the nanopipette is filled with and the tip immersed in an electrolyte and a voltage is applied between an electrode in side and an electrode outside of the nanopipette to generate an electric field at the tip. This field drives the molecule of interest through the nanopipette pore, resulting in a detectable pulse.

The present invention finds utility in healthcare applications, such as diagnostics. For example, the carrier molecules and methods of the invention may be used in the detection of analytes which are indicative of a disease or condition, e.g. biomarkers or toxins.

Thus, in some embodiments, the invention provides a method for diagnosing a disease or condition in a subject, the method comprising detecting and/or quantifying the presence of a target analyte (e.g. a biomarker) in a sample obtained from the subject.

The sample may be a liquid. In some embodiments the sample is a biological fluid, such as blood, plasma, serum, urine, sputum, saliva, or another fluid suspected to contain a particular analyte. However, it will be appreciated that the sample may be any type of sample in which the presence of target analytes may be detected. For example, the invention may be used to determine the purity, quality and/or safety of food or beverage samples, environmental samples, water samples, or chemical samples.

The subject may be a mammal, such as a mouse, rat, pig, horse, cow, goat, rabbit or primate. In some embodiments the mammal is a human.

It will be appreciated that the present invention is not limited to healthcare applications, but may also be utilised in any field in which the detection of an analyte is required, such as environmental analysis, water quality testing, quality control in food production, agriculture or related fields.

The target analyte may be any analyte of interest. It may be a biological analyte, or a chemical analyte. For example, the analyte may be a protein, peptide, antibody, cytokine, small molecule (e.g. a drug molecule), toxin, nucleic acid, polysaccharide, lipid, hormone or inorganic compound.

In a further aspect, there is provided a kit for the detection of a target analyte, the kit comprising:
a carrier molecule in accordance with the first aspect of the invention; and
instructions for use.

The kit may further comprise a nanopore. The nanopipette may be a nanopore as described herein. The nanopore may be a solid state nanopore. In some embodiments, the nanopore is provided in a nanopipette, e.g. a glass nanopipette.

The kit may further comprise one or more additional components selected from: an amplifier; a digitator; one or more electrodes; or a faraday cage.

In some embodiments the kit comprises a program for analysing the ion current signatures generated. Such a program may be provided in one or more electronic processors, or may be provided in a computer-readable storage medium (e.g., a non-transitory storage medium) that may comprise any mechanism for storing information in a form readable by a machine or electronic processors/computational device, including, without limitation: a magnetic storage medium (e.g., floppy diskette); optical storage medium (e.g., CD-ROM); magneto optical storage medium; read only memory (ROM); random access memory (RAM); erasable programmable memory (e.g., EPROM ad EEPROM); flash memory; or electrical or other types of medium for storing such information/instructions.

The carrier molecules and methods of the invention may be further used to quantify the amount of a target analyte in a sample.

In some embodiments the method is for quantifying the amount of target analyte present in the sample. In such embodiments, the method may comprise:
contacting the sample with a known concentration of carrier molecule; and
determining the ratio of occupied carrier molecules (i.e. carrier molecule-target analyte complexes) to unoccupied carrier molecules (i.e. carrier molecules to which no target analyte has bound).

Based on the ratio of occupied to unoccupied carrier molecules, and the known concentration of the carrier molecule added to the sample, the concentration of the target analyte can be calculated.

As described above, the presence or absence of the target analyte in the central void of the nucleic acid frame can be detected using a nanopore by observing the ion current signature as the carrier molecules pass through the nanopore. Unoccupied carrier molecules (i.e. with no target analyte bound to the binding moiety) produce a characteristic double peak. Provided that the size of the central void is suitably tailored to the size of the target analyte, occupied carrier molecules will produce a single peak. The frequency of the two characteristically different peaks can then be computed from the observation of a number of individual peaks to obtain an analyte-concentration dependent signal.

Thus, in some embodiments, determining the ratio of occupied carrier molecules to unoccupied carrier molecules comprises:
subjecting the sample to voltage-driven translocation through a nanopore; and
measuring the ion current signatures produced as the carrier molecules translocate through the nanopore,
   wherein the ratio of single peaks to double peaks in the ion current signatures is indicative of the ratio of occupied carrier molecules to unoccupied carrier molecules.

False positives, for example due to broken or truncated carrier molecules, can be excluded from the quantitative analysis by:
disrupting the structure of the carrier molecule;
subjecting the disrupted carrier molecules to voltage-driven translocation through a nanopore;
measuring the ion current signatures of the disrupted carrier molecules; and
determining the average peak amplitude and/or the average dwell time of the peaks in the ion current signatures.

Thresholds of peak amplitude and/or dwell time may be determined, which can then be used to exclude single peaks generated by the translocation of disrupted carrier molecules from subsequent quantitative analysis.

All three parameters of the ion current signature (peak shape, peak amplitude and dwell time) can therefore be used for the quantitative detection of a target analyte with a greater degree of confidence. The carrier molecule of the invention is thus advantageous in that the signal produced by broken carriers as they pass through a nanopore is substantially different in terms of dwell time, peak amplitude and peak shape to that produced by in-tact carriers, thereby enabling false positives to be filtered out.

Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, and with any aspect of the invention, unless such features are incompatible. For the avoidance of doubt, the terms “may”, “and/or”, “e.g.”, “for example” and any similar term as used herein should be interpreted as non-limiting such that any feature so-described need not be present. Indeed, any combination of optional features is expressly envisaged without departing from the scope of the invention, whether or not these are expressly claimed. The applicant reserves the right to change any originally filed claim or file any new claim accordingly, including the right to amend any originally filed claim to depend from and/or incorporate any feature of any other claim although not originally claimed in that manner.

Embodiments of the invention will now be described by way of example with reference to the accompanying drawings in which:

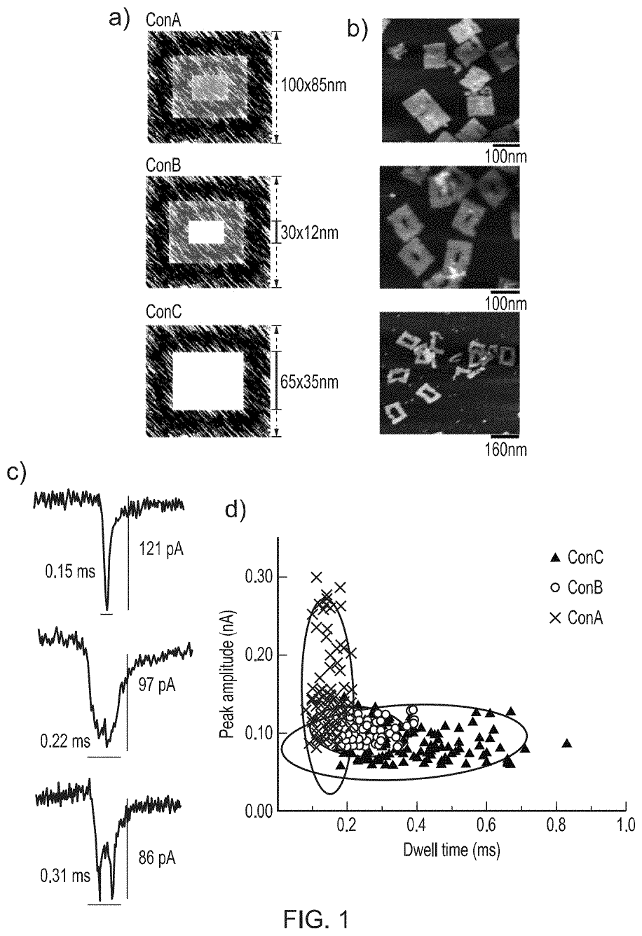

FIG. 1a is a schematic representation of DNA nanostructure design with different geometrical organization with ConA being a solid tile and ConB and ConC forming frames. All three structures exhibit similar external dimensions but varying internal voids;

FIG. 1b shows AFM micrographs of the nanostructures of FIG. 1a;

FIG. 1c shows the ion current signatures of the nanostructures of FIG. 1a

FIG. 1d shows a scatter plot of individual events, peak amplitude plotted against dwell time for ConA, ConB and ConC, overlaid with the 95% confidence eclipses;

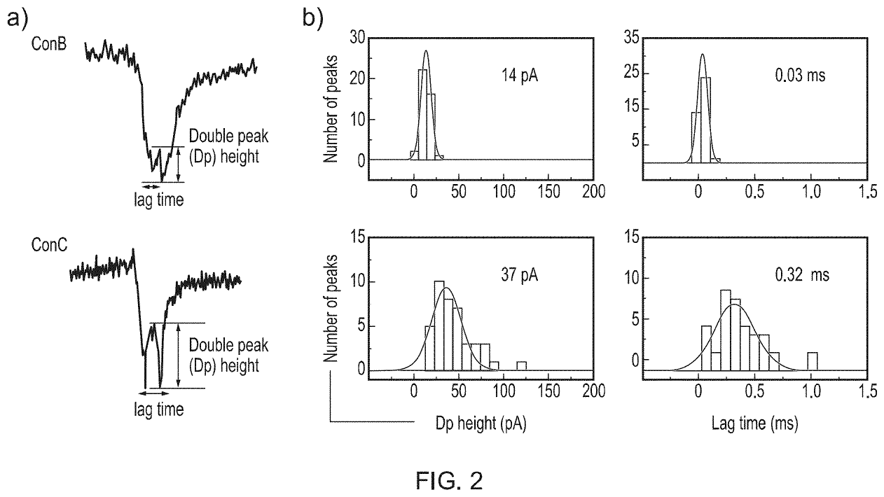

FIG. 2 shows lag time and double peak (Dp) height analysis for the two concentric square nanostructure featuring central cavities (ConB and ConC), with the mean values indicated;

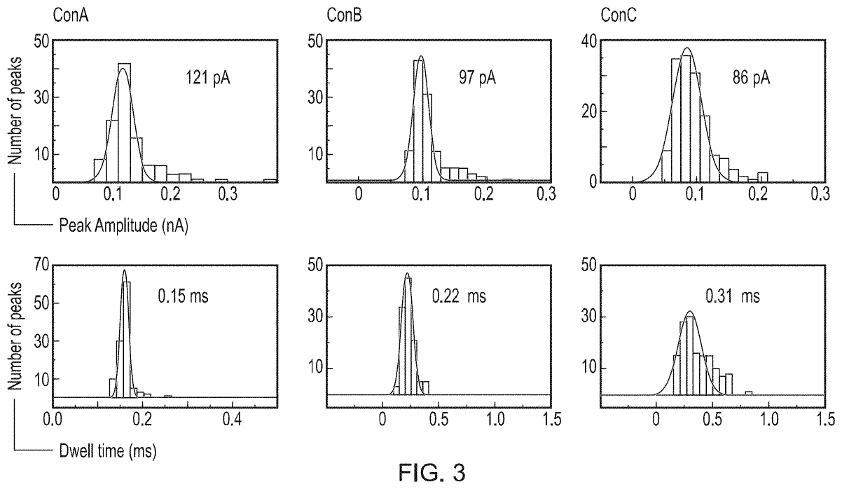

FIG. 3 shows individual peak amplitude and dwell time histograms of nanostructures ConA, B and C, indicating their mean;

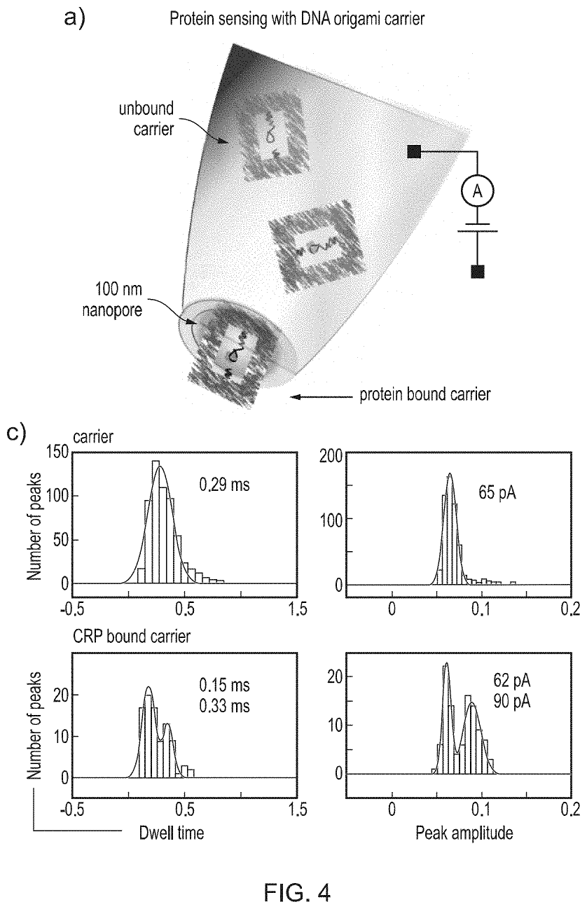

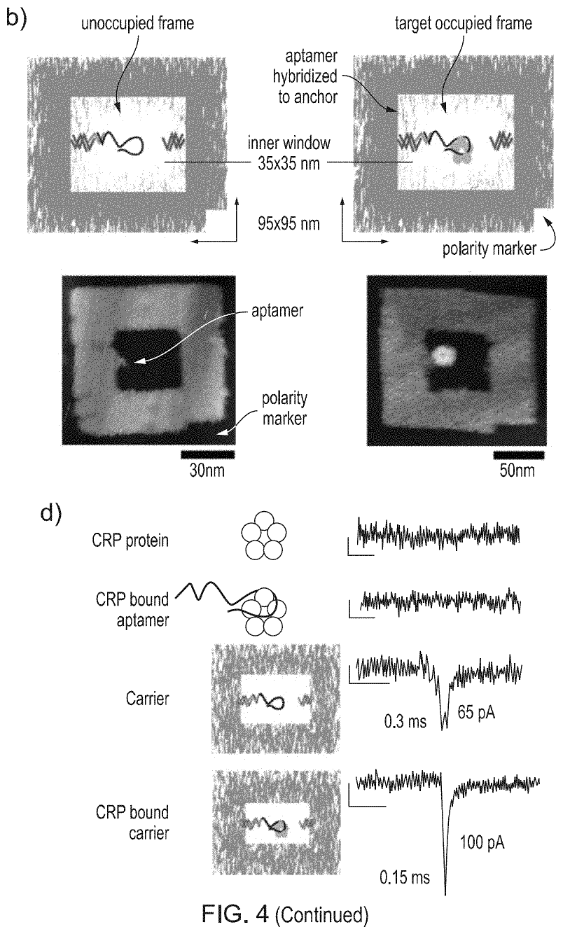

FIG. 4a is a schematic representation of the DNA nanostructure-based biosensing concept exploiting translocation through nanopipettes as the sensing mechanism;

FIG. 4b shows a schematic representation of the design and representative AFM micrographs of the unoccupied and occupied DNA origami carriers. The frame DNA nanostructure is approximately 95×95 nm in dimension with a 35×35 nm inner void. The structure comprises small nucleotide ‘anchors’ that protrude into the inner void which facilitate the incorporation of a DNA aptamer (hook shaped) via hybridization. The DNA carrier also includes an orientation marker (shown as a notch in the bottom right-hand corner);

FIG. 4c shows the peak amplitude and dwell time histograms of DNA frame carrier (9 nM) and carrier sample incubated with target CRP at ˜9 nM concentration;

FIG. 4d shows ion current signatures upon translocation of CRP molecules, CRP bound aptamer molecules, carrier molecules and CRP bound carrier molecules. The scale bars represent 20 pA in the y-axis and 1 ms in x-axis;

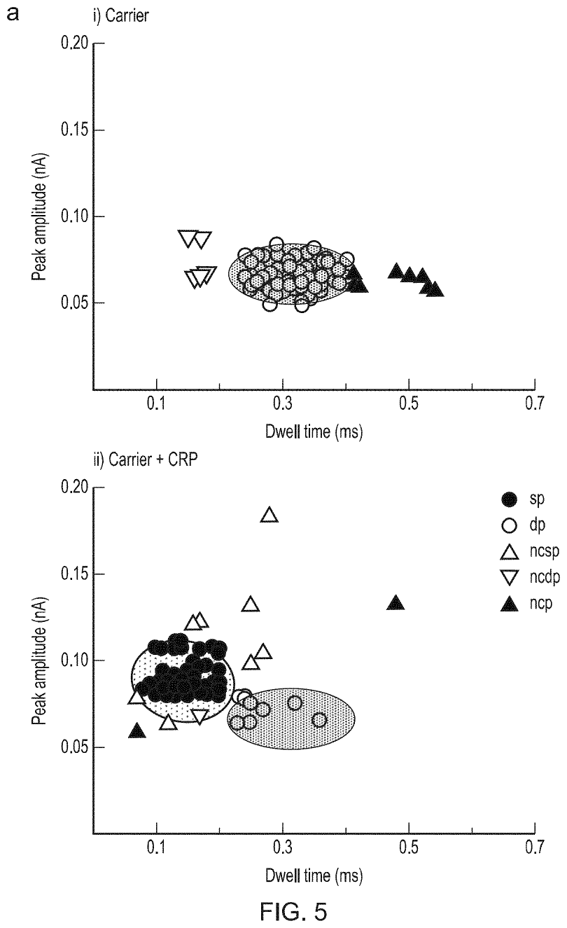

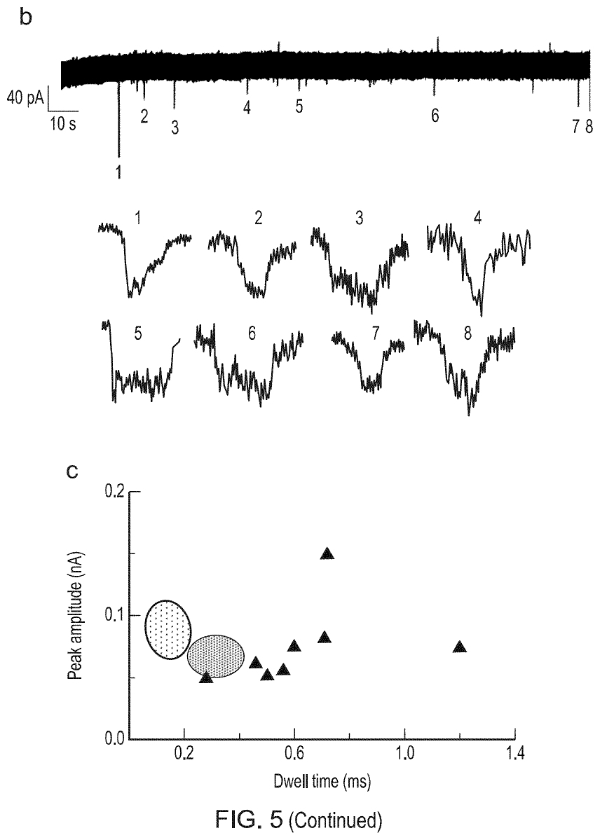

FIG. 5a shows scatter plots of ion current peaks for (i) unoccupied carriers and (ii) carriers incubated with a 10× excess of CRP (90 nM). The events that fall inside the 95% confidence ellipse are plotted as circles, the others as triangles. Only events that fall within the 95% confidence ellipse are considered double peaks. The same analyses was carried out for single peaks. Ion current events which resembled neither a double nor a single peak are shown as filled triangles and are excluded from the analysis;

FIG. 5b shows an ion current trace for broken carriers recorded for about 2 minutes;

FIG. 5c shows a scatter plot of peak amplitude versus dwell time for the broken carriers, with the 95% confidence ellipses overlaid;

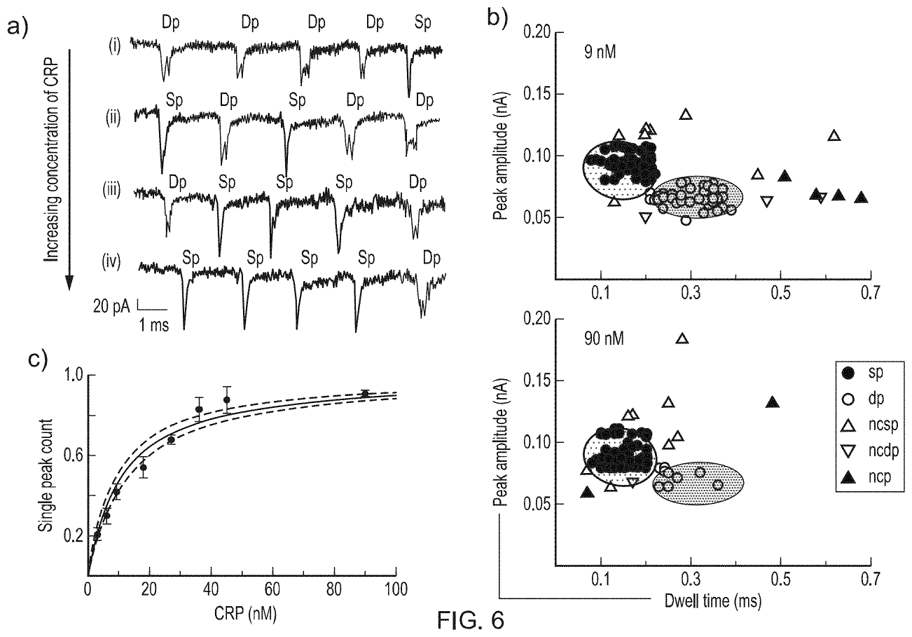

FIG. 6a shows a representative selection of ion current signatures observed for different CRP concentrations. The peak traces are stitched together from individual peaks of a longer trace to remove regions with no events for the purpose of illustration;

FIG. 6b shows plots of peak amplitude plotted against dwell time for a series of translocation events of carriers (9 nM) incubated with different concentrations of CRP. The unclassified events discarded from the quantitative analysis are represented as triangles. The plots are overlaid with 95% confidence eclipses;

FIG. 6c is a graph of the normalized single peak count, i.e. ratio of single peaks vs total classified peaks against CRP concentration. The data were fitted with Langmuir isotherm (solid line) an revealed an Kd of 11±2 nM. The dashed lines represent the confidence boundaries of the fit. The error bars denote standard deviation of translocation experiments conducted on different days using three different nanopipettes;

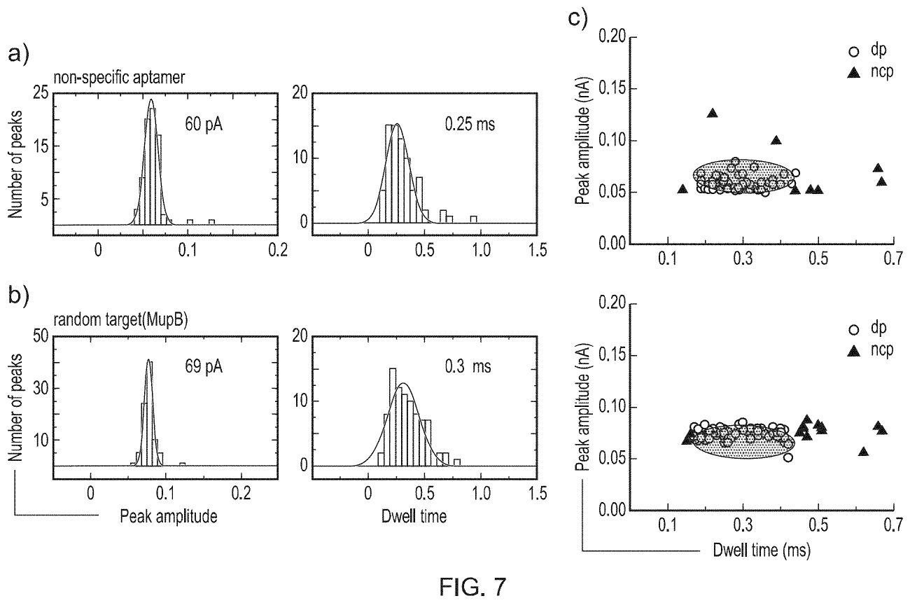

FIG. 7a shows histograms of dwell time and peak amplitude observed for a control experiment with a non-specific aptamer with CRP at 1:10 concentration. The control experiment exhibited a peak amplitude and dwell time mean of 69±5 pA and 0.3±0.13 ms that is similar to empty carriers indicating negative CRP binding to random aptamer sequence;

FIG. 7b shows histograms of dwell time and peak amplitude observed for a control experiment with a non-specific target protein, MupB at 90 nM and 9 nM of carrier concentration. The individual peak amplitude and dwell time histograms for the sample portrays a mean value of 60±7 pA and 0.25±0.1 ms that is again similar to empty carriers indicating negative non-specific target binding;

FIG. 7c is the scatter plot analysis, which scows the classification of the ion current events. No occupied carriers were detected, demonstrating the high specificity of the sensing approach;

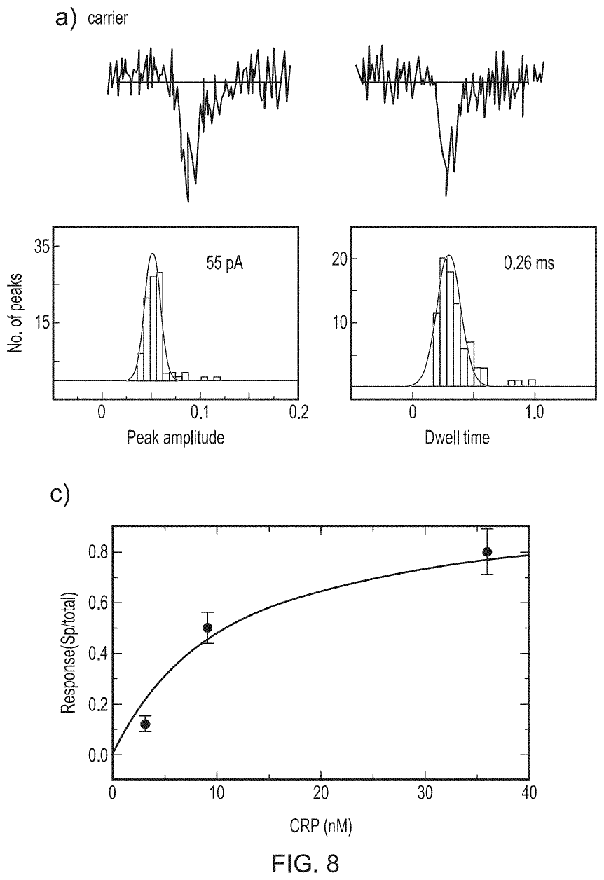

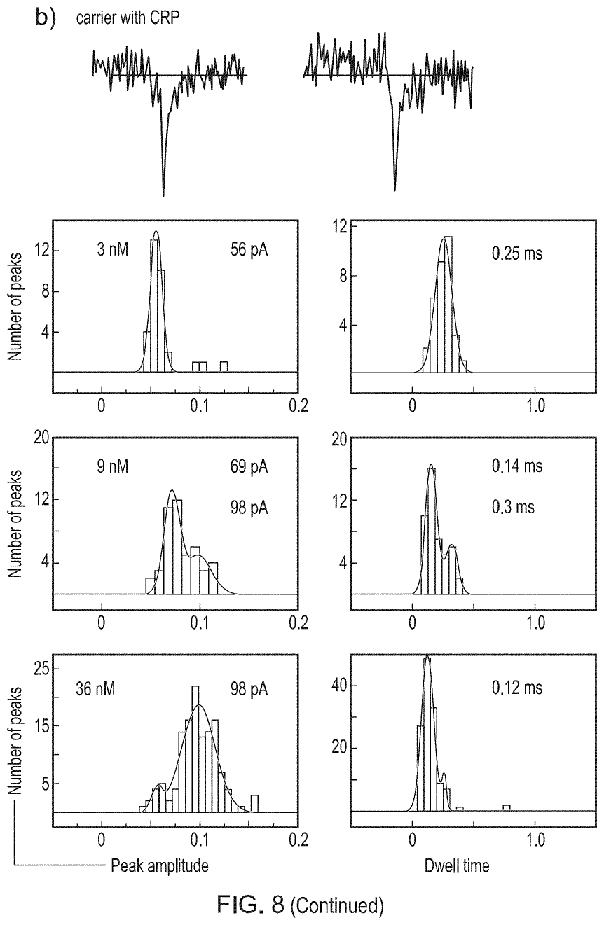

FIG. 8a shows representative ion current traces with peak amplitude and dwell time histograms of translocation experiments for unoccupied carriers in 5% plasma;

FIG. 8b shows representative ion current traces with peak amplitude and dwell time histograms of translocation experiments for carrier incubated with CRP for different concentrations of CRP in 5% plasma;

FIG. 8c shows a plot of the ratio of single peaks plotted against the respective CRP concentration in plasma. The data follows a Langmuir fit as shown by the curved line;

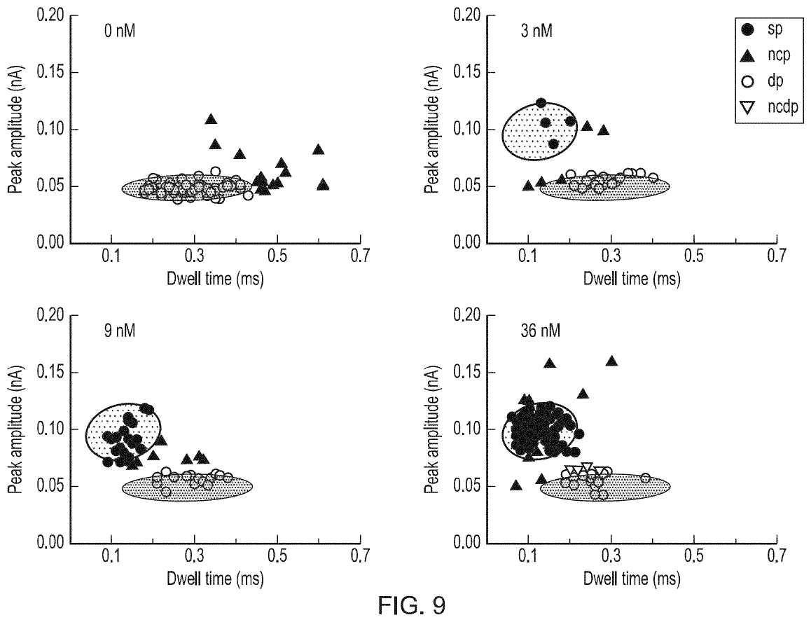

FIG. 9 shows scatter plots of peak amplitude versus dwell time for carriers (9 nM) subjected to four different concentrations of CRP (0 nM, 3 mM, 9 nM and 36 nM) in plasma. For 0 nM, no single peaks were observed, and the cluster of double peaks was used to define the 95% confidence interval for the unoccupied carrier. Similarly, the 95% confidence ellipse for the single peals was generated from the data of the highest CRP concentration;

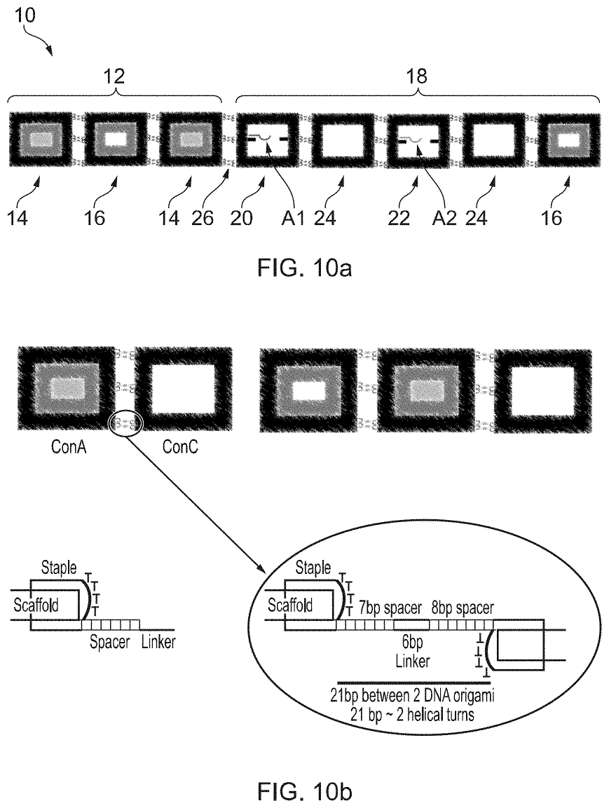

FIG. 10a is a schematic of a carrier molecule comprising a barcode for multiplexing;

FIG. 10b is a schematic of a linker strategy for joining subunits of a multiplex carrier molecule. The lines represent single stranded DNA and where they lie in parallel with another line this indicates base pairing between the two; and

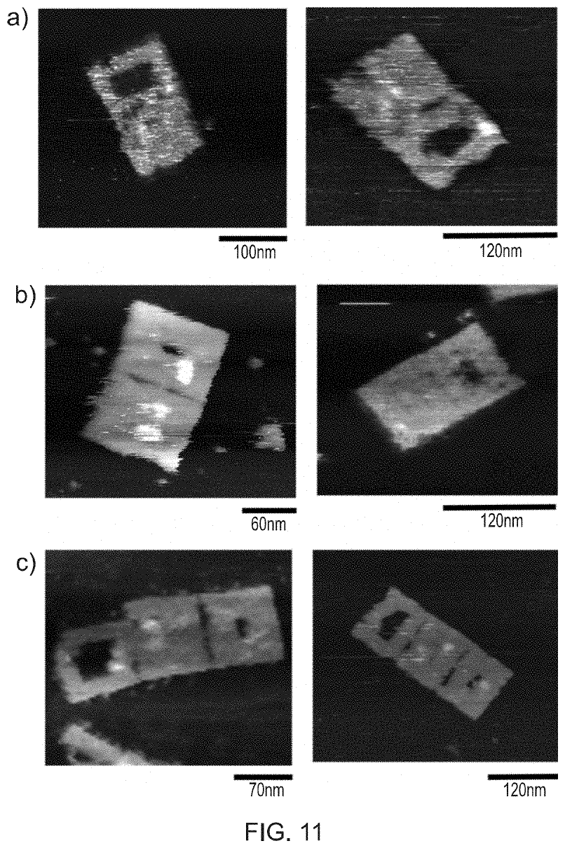

FIG. 11 shows atomic force microscopy images of example ribbons; including: a) a duplet formed from solid tile and large cavity frame-like subunits; b) a duplet formed from solid and small cavity frame-like subunits; and a) a triplet formed from large cavity frame-like, solid, and small cavity frame-like subunits.

### EXAMPLES

Materials and Methods

DNA Origami Design

The three concentric square origamis were designed using the custom made single-stranded DNA (ssDNA) scaffolds of 9073 bp, 8515 bp and 6307 bp whereas the DNA nanostructures used as carriers in this project were designed from 7249 bp m13mp18 ssDNA from Tillibit (Garching, Germany). The procedure for custom made scaffolds is provided below. All short staple strands for the respective nanostructure designs were purchased from IDT (Coralville, Iowa, USA).

All DNA origami nanostructures were designed using caDNAno software. For the origami folding, a mixture of ssDNA scaffold with a tenfold molar excess of the staple strands in the folding buffer containing 10 nM TrisAc (pH 7.4), 10 mM MgAc and 1 mM EDTA (Sigma Aldrich, USA) was heated to 95° C. and then cooled to room temperature with 1° C./minute decrease. The folded structures were then purified by removal of excess staples via a Sephacryl S400 (GE healthcare,UK) size inclusion column and eluted into the same folding buffer.

The aptamer sequence containing a modified end sequence with 5′ (aptamer 1) and 3′ (aptamer 2) amine attachment was incorporated into the carrier design in a second thermal annealing step (heating up to 35° C. followed by decrease to room temperature at 0.5° C. per minute) either in the presence of the staples before the purification step, or alternatively immediately after the purification step. Either way resulted in precisely folded frame DNA origami carriers inserted with a aptamer.

Specific hCRP aptamer 1:
(SEQ ID NO. 1)
NH2-AAGCCTTTATTTCAACGGCAGGAAGACAAACACGATGGGGGGGTA
TGATTTGATGTGGTTGTTGCATGATCGTGGTCTGTGGTGCTGT
Specific hCRP aptamer 2:
(SEQ ID NO. 2)
CGAAGGGGATTCGAGGGGTGATTGCGTGCTCCATTTGGTGTTTTTTTTT
TTTGCAAGGATAAAAATTT-NH2
Nonspecific random aptamer:
(SEQ ID NO. 3)
NH2-CTGAACAAGAAAAATAGCAGAACTTACGAGCCAGGGGAAACAGTA
AGGCCTAATTAGGTAAAGGAGTAAGTGCTCGAACGCTTCAGA

SPR Study

Aptamer-protein binding studies were carried out via an ESPRIT SPR instrument from Metrohm Autolab B.V (Utrecht, The Netherlands). For this, the end-modified aptamers attached with an amine group were anchored onto the gold SPR surface functionalized with a self-assembled monolayer (SAM) of C11PEG6COOH via EDC (1-ethyl-3-(3-dimethylaminopropyl) carbodiimide) NHS (N-hydroxysuccinimide) coupling chemistry. The gold SPR surface from XanTec bioanalytics was cleaned by sonication twice in acetone for 10 minutes and then immersed in an ethanolic 1 mM SAM solution containing 5% acetic acid and allowed to stand at room temperature for 48 hours. Following the incubation, the surface was rinsed with 100% ethanol, quickly dried with nitrogen and was mounted on the SPR system. To attach the DNA aptamers to the gold surface, the COOH-terminated SAM surface was activated with 50 mM EDC and 200 mM NHS in 100 mM MES buffer at pH5.5 for around 15 min. The surface was washed with the MES buffer, followed by a 30-minute incubation with 5 μM of DNA aptamer in 10 mM sodium acetate buffer at pH5.5. Any remaining activated COOH sites were quenched by exposing the surface to 100 mM ethanolamine in water for around 10 min. The surface was then washed with the binding buffer (10 mM TrisAc, 10 mM MgAc, 2 mM CaCl₂ and 1 mM EDTA in water) and then challenged with varying concentration of human CRP. The binding was measured as the change in resonance angle.

Generation of Concentric Squares Origamis

A modified phagemid (plasmid with m13 region) was used to create the three scaffolds of varying lengths to create the DNA origamis with similar outer but different cavity dimensions. The scaffolds required for the three DNA origamis (concentric squares (Con) A, B and C) were stitched together from two separate fragments derived from lambda phage DNA along with pBluescript II SK(+) phagemid (PBS). The PBS(+) are plasmids containing the phage origin of replication (f1 ori) and an antibiotic resistance gene which allows for sense strand rescue by a helper phage. The 9 kb custom scaffold required for the DNA origami ConA was created by inserting a 6.1 kb sequence, derived as two separate fragments from lambda phage, into the 2.9 kb phagemid. These two fragments (approximately 1.8 kb and 4.2 kb) were selected devoid of protein coding regions to eliminate any interference in the phage growth and downstream process.

The three fragments that make up the scaffold—referred to as f1, f2 and f3—were PCR amplified from lambda phage DNA and the PBS plasmid, respectively, using appropriate primers. The primers used in the amplification procedure were designed to incorporate the restriction sites EcoRI and NspI flanking f2, and NcoI and NspI flanking f3. The two fragments were then restriction digested with the respective enzymes to form compatible overhangs, and then ligated to yield one long fragment (f23). Fragment f1 was then combined with fragment f23 using HiFi DNA assembly cloning (NEB, UK), resulting in the double stranded circular f123 dsDNA.

Subsequently the circular dsDNA (f123) was chemically transformed in competent E. coli cells to produce the custom scaffold. The transformed cells with the f123 plasmid were selected using their antibiotic resistance gene and recovered with a miniprep to be co-transformed with helper plasmids (m13cp cells) to produce ssDNA f123. The final ssDNA f123 product constituted 9073 bp and made up the scaffold for the largest DNA construct ConA.

The scaffolds for the two smaller DNA constructs, ConB and ConC, were produced by PCR amplification of the double stranded f123 as template. As before the primers were designed to incorporate the restriction digest site KasI for the product ConB and BmtI for the product ConC. Subsequent restriction and ligation followed by similar co-transformation protocol with helper plasmids as above resulted in 8515 bp and 6307 bp ssDNA scaffolds for Con B and C, respectively. In the first instance, the scaffolds were produced in 5 ml cultures later on followed by large-scale scaffold harvesting by scaling up to 1-litre cultures. The cultures were harvested by separating the bacteria from the phage and subsequent phenol chloroform DNA extraction.

Nanopipette Fabrication and Ion Current Measurements

The nanopipettes with ˜100 nm pore diameters were fabricated from glass capillaries of 0.5 mm inner diameter (QF100-50-7.5, World precision Instruments, UK) using a Sutter instrument model P-2000 laser puller. The pulling protocol comprised two separate lines with the parameters HEAT 575 FIL 3 VEL 35 DEL 145 PULL 75 and HEA T900 FIL 2 VEL 15 DEL 128 PULL 150. The protocol revealed highly consistent glass nanopipettes with a standard deviation of less than ±12 nm with pipettes pulled on different days. An Ag/AgCl wire (0.25 mm diameter, Sigma Aldrich, UK) was inserted into the nanopipettes as a working electrode.

For the translocation experiments the nanopipettes with the working electrode were filled with the translocation buffer (0.1 M KCl with 10 mM TrisAc, 10 mM MgAc, 2 mM CaCl₂) and 1 mM EDTA) containing the DNA origami and analyte where applicable at a final concentration of 500 pM. The Mg is required to maintain the stability of the DNA origami, and the Ca to match the buffer conditions used to select the DNA aptamers employed as binding moieties. The grounded counter electrode was immersed in a 0.1 M KCl solution completing the circuit. On application of a negative potential to the working electrode inside the nanopipette, DNA origami from inside the nanopipette are translocated out into the electrolyte solution resulting in a modulation of the ion current. Ion current data were acquired using an Axon instruments-patch clamp system (Molecular devices, USA). Measurements were recorded using the Axopatch 700b amplifier, and the data were acquired at a rate of 100 kHz and 20 kHz low pass filtered. Initial data analysis was carried out with a custom MATLAB script (provided by Prof. Joshua Edel, Imperial College, London, UK) and further data analysis was done using Pro FIT(QuanSoft, Switzerland).

AFM

DNA origami samples were deposited on freshly cleaved mica discs for 10-15 minutes at room temperature and topped up with scanning buffer containing 10 mM TrisAc (pH 7.4) and 10 mM MgAc, 1 mM EDTA. For observing protein binding to carriers, 2 mM CaCl₂ was included to the scanning buffer similar to the nanopipette translocation experiments. The DNA origami samples were imaged using a Bruker Dimension Fastscan (Santa Barbara, Calif., USA) with Fastscan D Si₃N₄ cantilevers containing a Si tip in tapping mode in liquid. Images were obtained with scan rates of 20 kHz (256×256 pixels).

### Example 1: Concentric Squares

It was hypothesized that if a DNA origami is designed to contain a cavity in the center into which the analyte of interest can bind specifically, the presence or absence of the analyte from the origami can be detected using a nanopore by observing the characteristic translocation ion current peak. The frequencies of the two characteristically different peaks can be computed from the observation of a large number of individual peaks to obtain an analyte-concentration dependent signal.

To gain further insight into the design parameters of the cavity in the DNA origami, we designed three different DNA nanostructures with identical outer but variable cavity dimensions. The three DNA nanostructures resemble a set of three concentric squares and are referred to as ConA for a solid nanostructure (100 nm length×85 nm height), ConB and ConC for a nanostructure of identical outer dimension but containing a central cavity of 30 nm length×12 nm height and 65 nm length×35 nm height, respectively, as shown in FIG. 1a.

The concentric square nanostructures were all designed to be folded according to established DNA origami principles. The three tiles are made from the same DNA, i.e. the same set of DNA oligonucleotides staples and DNA scaffold by simply shorten in the scaffold and leaving out the appropriate staples to generate the cavities of different size. For example, the scaffold DNA used to assemble ConB was the same as the one used for assembling ConA but with the part that folds the central part removed. This ensures that as much as possible of the structure remained identical between DNA origami while varying the cavity dimension so we can directly correlate the ion current signature to the cavity volume.

The folded DNA nanostructures were imaged with Atomic Force Microscopy (AFM) to confirm successful assembly. Representative AFM images are shown in FIG. 1b and it can be seen that all three structures were formed as intended. ConA was found to be 107×86 nm, and ConB and ConC with similar external dimensions of 107×86 nm and with a cavity of 29×11 nm and 65×38 nm, respectively.

Nanopore translocation study for the individual concentric square samples was conducted through glass nanopipettes with ˜100 nm pore diameter. The nanopipettes were fabricated via laser pulling and showed a resistance of 86±10 MΩ in 0.1 M KCl. Translocation of the concentric squares was carried out by loading a sample solution at a concentration of 500 pM into the nanopipette and recording the ion current while applying a constant voltage of −350 mV across the nanopore.

FIG. 1c shows representative ion current signatures for individual nanostructures translocating through the nanopore. As a cavity is introduced in the nanostructure the observed peak structure changes from a single peak (no cavity present) to a double peak (cavity present), with the double peak becoming more pronounced with increasing cavity size (double peak height of 37 pA for ConC vs 14 pA for ConB, FIG. 2). A more detailed quantitative analysis of at least 100 ion current peaks for each nanostructure shows that the average peak amplitude and dwell time differ significantly for different nanostructures (FIG. 3).

ConC (large cavity) translocates through the nanopore significantly slower (dwell time=0.31±0.14 ms) and leads to a smaller ion current increase (peak amplitude=86±19 pA) when compared to that of ConB (small cavity; dwell time=0.22±0.06 ms and peak amplitude=97±13 pA). In contrast, the ConA (no cavity) nanostructure causes the largest ion current increase (peak amplitude=121±23 pA) with the shortest dwell time (0.15±0.01 ms). When taken together, these parameters allow us to identify distinct populations (FIG. 1d).

### Example 2: DNA Nanostructures as Translocation Carriers in Biosensing

The relationship of translocation ion current peak signature to cavity geometry within these DNA nanostructures provides a unique opportunity to detect the presence of a much smaller molecule. If a binding moiety specific to the molecule of interest is placed within the cavity of the DNA nanostructure, the binding of a target analyte to the binding moiety partially fills the cavity which in turn results in a characteristic change to the ion current signature (FIG. 4a).

In order to explore this and as a proof of principle, we demonstrate the quantitative detection of C-reactive protein (CRP), which is an established inflammation biomarker. In a healthy adult the median CRP concentration is 0.8 mg/L, and its concentration in blood exceeds 1 mg/mL (8 μM) as a result of an inflammatory response. CRP exists as a pentamer with a molecular weight of approximately 125 kDa and a size of around 11 nm. We designed a DNA nanostructure with a central cavity large enough for a CRP molecule to fit in but at the same time small enough such that the presence of CRP in the cavity will make a measurable difference to the translocation ion current. As informed by the study with concentric squares (Example 1), the nanostructure was designed with a central cavity of 35 length×35 nm width to provide a robust double peak signature (FIG. 4b).

An internal anchor stub was introduced within the central cavity such that a specific capture moiety can be placed inside the cavity via DNA hybridization (FIG. 4b). In order to enable selective CRP detection, we chose two well-characterized CRP DNA aptamers (aptamer 1 and aptamer 2) from the literature as potential capture moieties. We note that while the sequences of the DNA aptamers employed here are the same as the ones published, for this application the aptamers have to be extended at their 5′ or 3′ end, respectively, so they can be hybridized into the cavity. To ensure the DNA aptamers are still performing as intended, we confirmed CRP binding to the extended aptamers by Surface Plasmon Resonance (SPR). Both DNA aptamers continued to bind CRP as expected. Aptamer 1 showed faster binding kinetics compared to aptamer 2 and hence was selected for the detailed demonstration of the biosensor concept.

A notch was also included at one corner of the DNA origami to act as a polarity marker to identify the orientation of the origami in AFM images (FIG. 4b). The complete DNA nanostructure and aptamer assembly is referred to as the carrier.

The performance of the DNA aptamer within the context of the carrier (i.e. aptamer hybridized within the DNA nanostructure, FIG. 4b) was investigated by gel retardation assay. A 9 nM solution of the carrier was incubated with increasing concentrations of CRP (9, 18, 27, 36 nM) in the translocation buffer (0.1 M KCl containing 10 mM MgAc, 2 mM CaCl₂ and 10 mM TrisAc and 1 mM EDTA) at room temperature for 30 minutes. The gel showed a concentration-dependent shift of the carrier bands, which demonstrates successful CRP binding to the carrier. CRP (36 nM) binding to the carrier (9 nM) was further confirmed by AFM under similar buffer conditions as stated above (FIG. 4b), where the CPR is clearly observed in the cavity bound to the aptamer which is opposite the inbuilt polarity marker.

To exploit the DNA nanostructure carrier in single-molecule nanopore sensing, the occupied and unoccupied carriers need to have unique fingerprints (composed of dwell time, amplitude and shape of the ion current peak) which relate to the presence of CRP within the carrier. Nanopore translocation studies of the carriers (at 500 pM concentration) using glass nanopipettes were carried out as above. The quantitative analysis of more than 100 translocation peaks revealed a peak amplitude of 65±6 pA and dwell time of 0.29±0.1 ms (FIG. 4c, top) which are comparable with previous observations of similar-sized DNA nanostructures containing cavities.

Similarly, to analyse the CRP-occupied carriers we used an equimolar solution of CRP and carriers at 9 nM with the aim of having a mixed population of occupied and unoccupied carriers. The solution was incubated at room temperature for 30 min in translocation buffer and subjected to nanopipette translocation at 500 pM concentration, which led to a mixed population of single and double peaks in the ion current. The analysis of the peak amplitudes and dwell times of the two classes of peaks revealed a bipolar distribution. The double peaks had an average peak amplitude of 62±4 pA and dwell time of 0.33±0.03 ms (FIG. 4c, bottom; n>100). In contrast, the analysis of more than 50 single peaks revealed an average dwell time and peak amplitude of 0.15±0.05 ms and 90±9 pA, respectively, which demonstrates a substantial shift in both quantities as a result of CRP binding (FIG. 4c, bottom). We note that the lower peak amplitude and the longer dwell time are very similar to the ones observed for the unoccupied carriers.

Together with the results of the concentric squares study which showed that the peak shape changes from a double peak to a single peak upon filling in the central cavity, we speculate that the single peak events correspond to occupied carriers, i.e. carriers with a CRP bound to the specific aptamer. Furthermore, the double peaks are accounting for approximately 30% of the total number of observed events. This is in line with the percentage of occupied carriers that would be expected based on the Kd obtained from the SPR experiments, which suggests that the percentage is concentration dependent as expected.

However, in order to use this approach for high-sensitivity biosensing, a reliable way of classifying the different ion current events is required. FIG. 5a(i) shows the scatter plots of the peak amplitudes versus dwell times of the ion current peaks observed for the translocation of unoccupied carriers. All events which resemble the shape of double peaks are shown as open circles and open, inverted triangles. Filled triangles represent events that cannot be classified. To eliminate outliers and to ensure robust classification, ion current events will only be classified as a true double peak representing an unoccupied carrier (indicated by open circles) if the measured peak amplitude and dwell time fall within the 95% confidence ellipse, which is indicated in the figure. Peaks which fall outside of this boundary are indicated by open inverted triangles and will not be considered as events representing unoccupied carriers. Similarly, FIG. 5a(ii) shows the scatter plots of the peak amplitudes versus dwell times of the ion current events observed for the translocation of carriers incubated with ten times excess of CRP expected to lead to the majority of carriers being occupied. All events which resemble the shape of double peaks are shown as open circles and open, inverted triangles. Single peaks are shown as filled circles and open triangles. All other events are shown as filled triangles, indicating that they cannot be classified. As above, to ensure a robust classification for single peaks to represent CRP-occupied carriers, the 95% confidence ellipse (indicated as a light grey (left-hand) ellipse) is employed as an in-out filter. Only single peaks which fall within this 95% confidence area are considered as resulting from the translocation of a CRP-occupied carrier (indicated by filled circles), all other single peaks are considered unclassified (indicated by open triangles). To classify the double peaks, and thereby establishing the number of events representing unoccupied carriers, the confidence ellipse from panel (i) is indicated (as the dark grey (right-hand) ellipse), and now only double-peak events which fall within this area are considered as representing unoccupied carriers (open circles), while the ones outside this area are dismissed (open, inverted triangles).

This now enables the classification of observed ion current peaks into three categories, double peaks representing unoccupied carriers, single peaks representing CRP-occupied carriers, and unclassified peaks which resemble neither a double nor a single peak. This multi-parameter classification allows the discarding of ambiguous translocation events, for example resulting from broken carriers, in a robust way. Such events would likely resemble single peaks and hence represent false positives. To illustrate this, the DNA nanostructures were deliberately disrupted by incubating in 10 mM CaCl₂ for 30 min to substitute the constituent Mg²⁺ with Ca²⁺ prior to translocation. AFM micrographs demonstrated that the carriers had been degraded significantly. During the translocation experiments only very few events were recorded, and the peak amplitude vs dwell time scatter plot shows that none of the recorded peaks fall within the relevant confidence ellipse (FIGS. 5b and c), demonstrating the robustness of the classification approach. As such, the counting of false positives from broken or truncated carriers in the sample is limited effectively by the filtering of the single peaks via the classification procedure discussed above. We note that the small concentration of Ca²⁺ in the translocation buffer does not affect the carriers significantly. Even after incubation of the carriers for 4 hours in the translocation buffer, only a small number (approximately 10%) of the translocation events would be classified as CRP-occupied carries with the above classification method.

Importantly, due to the large dimensions of the nanopipette pore (100 nm) compared to the diameter of CRP (11 nm), the translocation of CRP alone does not lead to a detectable ion current signature and hence cannot be detected by our nanopipette sensor (FIG. 4d). Similarly, any other molecules in the solution would also not lead to any signals, thereby neither contributing noise nor false negatives or positives.

### Example 3: Quantitative Single Molecule Biosensing

To demonstrate quantitative sensing using the translocation of carriers with specific DNA aptamers, and the concept of counting single individual carrier molecules classified through the three-parameter approach (peak dwell time, amplitude and shape), we analysed the ion currents of a range of translocation experiments at different CRP concentrations.

FIG. 6a shows a collection of representative ion current peaks for a range of different CRP concentrations. For each concentration, the observed ion current events were classified as described above. Where single or double peaks did not satisfy the filtering criteria they were marked as ‘unclassified’ and were not taken into account for the concentration analysis (FIG. 6b). Such unclassified peaks represented between 9-36% of the total number of events in a 2-minute trace.

FIG. 6c shows the normalized single peak count, i.e. classified single peaks vs total number of classified peaks, for different concentrations of CRP from 3 nM to 90 nM. As expected, the normalized single peak count increases with increasing CRP concentration. The data were fitted with a Langmuir isotherm, using the dissociation constant K_{d} as the only fitting parameter, and the result is shown as a solid line. The K_{d} obtained from the fit is 11±2 nM.

To investigate the specificity of our sensing system, and in particular of the carriers to the CRP target, a random DNA sequence was selected to act as a non-specific aptamer and the translocation ion current was measured for carrier concentration of 9 nM and CRP concentration of 90 nM, i.e. the highest concentration reported in FIG. 6. The analysis of the ion current events is shown in FIG. 7. A single distribution of peak amplitudes and dwell times with averages of 69±5 pA and 0.3±13 ms, respectively, were found, consistent with the values measured with unoccupied carriers. The scatter plot clearly shows that only double peaks were identified, and no single peaks, demonstrating that a non-specific aptamer does not lead to any detection signal.

Furthermore, the CRP-specific carrier (at a 9 nM concentration) was subjected to 90 nM of a control protein of similar size as CRP (MupB), and the results of the ion current analysis is shown in FIG. 7. Similar to the non-specific aptamer, single distributions of dwell time and peak amplitudes (averages of 0.25±0.1 ms and 60±7 pA) were observed which are in line with those for unoccupied carriers. The scatter plot clearly shows that no single peaks were identified (n>50) demonstrating that no MupB bound to the CRP-carriers.

Using the three-parameter classification (amplitude, dwell time and ion current signature) quantitative detection down to 3 nM CRP in ˜5 μl sample volume was achieved within a 2-minute sampling window.

Importantly, a similar study but with a different CRP-specific aptamer (aptamer 2) incorporated into the carrier was carried out. Very similar results to the carrier version with CRP aptamer 1 were obtained, demonstrating the robustness of the biosensing approach.

For applications in clinical diagnostics, it is important that quantitative detection of analytes such as CRP can also be performed in complex biological fluids. To demonstrate the performance of this sensor system with such fluids, nanopipettes were filled with solution of 5% human plasma diluted in 0.1 M KCl and spiked with the desired concentration of CRP ranging from 3 nM to 36 nM. Similar to the situation in buffer, the carriers in 5% plasma without CRP produced ion current double peak events, but with a slightly lower dwell time of 0.26±0.06 ms and peak amplitude of 55±6 pA (FIG. 8a, n>50). In contrast, the peak characteristics for occupied carriers, i.e. the single peaks observed in 5% plasma spiked with CRP, were found to be consistent between experiments in 5% plasma (peak amplitude 0.12±0.04 ms and dwell time 98±1 pA, n>30, FIG. 8b) and buffer (0.15±0.05 ms and 90±9 pA).

The different ion current events observed during a translocation experiment with 5% plasma can be classified into single peaks, double peaks and unclassified peaks in the same way as in buffer. FIG. 9 shows the scatter plots for peak amplitude versus dwell time for all CRP concentrations with the single and double peaks which were selected as representing CRP-occupied and unoccupied carriers, respectively, indicated in the same way as for the buffer experiments.

The normalised single peak count, i.e. the ratio of single peaks vs total classified peaks, is shown in FIG. 8c as a function of CRP concentration. Similar to the results in buffer, the normalized single peak count increases with CRP concentration, and follows a similar behavior. The limit of detection is estimated to be 9 nM (compared to 3 nM in pure buffer). We note that the number of unclassified events observed for various CRP concentrations in plasma samples were similar (9 to 25%) to those observed in pure buffer.

### Example 4: Carrier Multiplexing and Identify Barcodes

In order to multiplex the biosensing technology we can form ‘ribbons’ or polymers through the connection of multiple DNA nanostructures together. These ribbons may contain two functional elements; 1) an analyte detection region and 2) a barcode region.

The analyte detection region comprises a structure which takes the form of the carrier molecules already described, comprising a nucleic acid frame and a binding moiety. The empty or unoccupied frame produces a double peak in the ion current signal, whereas the presence of a bound analyte produces a single peak.

The barcode region is for identifying the ribbon, and it is this region that enables the detection of multiple analytes in a single sample. The barcode region makes use of the identified relationship between ion current signature and nanostructure geometry: For example, a solid tile structure produces a single peak whereas a frame-like structure produces a double peak. A finer implementation of this includes variations of frame cavity sizes to provide additional signals. The barcode region may comprise two or more subunits attached together in a specific order to provide a unique ion current fingerprint. These arrangements are inherently modular and therefore provide a flexible and economic approach.

FIG. 10a shows an embodiment of a multiplexed ribbon structure 10. An identity barcode region 12 is created using a specific combination of solid nucleic acid tiles 14 and a nucleic acid frame 16. The arrangement of solid-frame-solid would give an ion current fingerprint of single peak-double peak-single peak.

The barcode region 12 is attached to a detection region 18 comprising a first detection frame 20 containing a first aptamer A1, and a second detection frame 22 containing a second aptamer A2. In the embodiment shown the second aptamer A2 is specific for a different analyte to the first aptamer A1, although it will be appreciated that in alternative embodiments the aptamers may be the same. Three further nucleic acid frames 16, 24, each lack a binding moiety, make up the detection region 18. However, it will be appreciated that this approach is inherently modular and the nanostructure subunits can be rearranged in any fashion to provide unique barcode/analyte ribbons for the desired purpose.

The frames and tiles of the ribbon structure 10 shown in FIG. 10a are joined together by spacers 26.

The attachment of the DNA nanostructure subunits together into ribbons can be achieved through the interaction of protruding DNA linkers. FIG. 10b shows an example of how this may be implemented, but it will be appreciated that the attachment of adjacent structures using DNA linkers could be achieved in a number of different ways. The specificity of these interactions and therefore which subunit binds to which is dictated by the complimentary base pairing of specific DNA sequences at the extremity of these linkers. The DNA sequences used to specify these interactions are variable and tuneable. This allows the specificity of the interface to be designed and the strength of the interaction to be tuned (i.e. the melting temperature of the collective DNA pairings). This interaction strength is also tuneable by changing the number of DNA linkers (e.g. which, in some embodiments, could be from 1 to 6) between the subunits.

### CONCLUSIONS

We have demonstrated an alternative carrier molecule approach using a nucleic acid frame, specifically a DNA origami nanostructure, that is both selective and sensitive to the target analyte of interest. By utilizing the definite structural property of DNA origami towards translocating ion current, we employed frame DNA nanostructures in combination with glass nanopipettes as a biosensing platform that can detect and quantify single analyte molecules. We showed that by folding a long linear DNA, which is often the carrier of choice for nanopore experiments, many of the setbacks could be overcome. The specific ion current signature of the DNA origami carrier not only removes false positive incidents due to knots and folds but also eliminates the ‘tail to head’ and ‘head to tail’ events which are encountered with modified long linear DNA strands with respect to its translocation direction. This means our carriers exhibit a similar peak amplitude and dwell time translocation data that can be further verified with the specific ion current signature rather than the various ion current conductance events produced for linear DNA.

Moreover, the spatial addressability of the carriers of the present invention facilitates the incorporation of binding moieties such as aptamers in the DNA nanostructures at specific positions, that can influence the ion current.

Additionally, the change in ion current signature with respect to the position of the target analyte (CRP) provided a visual indication (double peak to single peak) towards target capture, in addition to the peak amplitude and dwell time difference. Through this three-factor technique we could quantitatively detect single analyte molecules in low concentrations high specificity. Importantly, the experiments demonstrated the ability of the carriers to detect the target analyte in complex plasma samples with similar sensitivity and signal to noise as seen in simple KCl buffer.

Yet another advantage of a nucleic acid frame carrier is that target capture can be visualized using microscopy techniques. Furthermore, the carrier molecule could be easily modified for multiplexing, enabling the detection of multiple target molecules simultaneously.

1. A carrier molecule for the detection and/or quantification of a target analyte, the carrier molecule comprising:
a molecular frame which defines a central void; and
a binding moiety which is specific for the target analyte, wherein the binding moiety is bound to the frame and positioned such that the target analyte, when bound to the binding moiety, is located in the central void.

2. The carrier molecule according to claim 1, wherein the carrier molecule is for carrying the target analyte through a nanopore.

3. The carrier molecule according to claim 1 or claim 2, wherein the molecular frame is formed from a nucleic acid, protein, peptide, or a mixture thereof.

4. The carrier molecule according to any one of claims 1 to 3, wherein the molecular frame is formed partially or entirely from DNA.

5. The carrier molecule according to any one of claims 1 to 4, wherein the molecular frame is formed by DNA or protein origami.

6. The carrier molecule according to any preceding claim, wherein the molecular frame is rectangular, square, circular, oval, triangular, trapezoid, rhomboid, pentagonal, hexagonal, octagonal, kite-shaped or irregular in shape.

7. The carrier molecule according to any preceding claim, wherein the molecular frame has a thickness of no more than about 20 nm.

8. The carrier molecule according to any preceding claim, wherein the molecular frame is substantially 2-dimensional.

9. The carrier molecule according to any preceding claim, wherein the molecular frame has a single conformation.

10. The carrier molecule according to any preceding claim, wherein the molecular frame is substantially rigid.

11. The carrier molecule according to any preceding claim, wherein the central void is sized such that it is capable of receiving the target analyte therein.

12. The carrier molecule according to any preceding claim, wherein the carrier molecule is configured such that:
in the absence of a bound target analyte, the carrier molecule produces a double peak in an ion current signature upon translocation of the carrier molecule through a nanopore; and
when a target analyte is bound by the binding moiety and located in the central void of the carrier molecule, a single peak in the ion current signature is generated upon translocation of the carrier molecule through a nanopore.

13. The carrier molecule according to any preceding claim, wherein the carrier molecule is configured such that the target analyte, when bound to the binding moiety, substantially fills or occludes the void.

14. The carrier molecule according to any preceding claim, wherein the molecular frame is configured such that at least a portion of a target analyte, when bound by the binding moiety, extends outside of the frame.

15. The carrier molecule according to any preceding claim, wherein the binding moiety comprises an aptamer, an affimer, an antibody (or a derivative or fragment thereof) a molecularly imprinted polymer (MIP) or a nucleic acid-protein fusion molecule.

16. The carrier molecule according to any preceding claim, wherein the binding moiety is bound to the frame via an anchor moiety.

17. A carrier molecule for the detection and/or quantification of a target analyte in a multiplex system comprising:
a detection region comprising at least one detection frame, which is a molecular frame which defines a central void, and a binding moiety which is specific for the target analyte, wherein the binding moiety is bound to the frame and positioned such that the target analyte, when bound to the binding moiety, is located in the central void; and
a barcode region attached to the detection region, wherein the barcode region comprises at least one nanostructure, wherein the nanostructure is a molecular tile or a molecular frame which lacks a binding moiety.

18. The carrier molecule according to claim 17, wherein the carrier molecule is defined according to any one of claims 2 to 16.

19. The carrier molecule according to claim 17 or claim 18, wherein the barcode region comprises a plurality of nanostructures arranged in series.

20. A composition comprising a carrier molecule according to any one of claims 1 to 19 in solution.

21. The use of a carrier molecule according to any one of claims 1 to 19, for detecting and/or quantifying the presence of a target analyte in a sample, said use comprising translocating the carrier molecule through a nanopore.

22. A complex comprising a carrier molecule according to any one of claims 1 to 19, bound to a target analyte.

23. A method for detecting and/or quantifying the presence of a target analyte in a sample, the method comprising:
contacting a carrier molecule according to any one of claims 1 to 19 with the sample; and
detecting the presence of a carrier molecule-target analyte complex.

24. The method according to claim 23, wherein detecting the presence of the carrier molecule-target analyte complex is carried out by voltage-driven translocation of the complex through a nanopore.

25. The method of claim 24, wherein a change in the ion current signature relative to the carrier molecule alone, is indicative of the presence of the target analyte in the sample.

26. The method of claim 25, wherein the change in the ion current signature is a change in the peak shape, the peak amplitude and/or the dwell time.

27. The method of claim 25 or claim 26, wherein a change in the peak shape from a double peak to a single peak is indicative of the presence of the target analyte in the sample.

28. The method according to any one of claims 24 to 27, wherein the nanopore is sized so as to avow the passage of a single carrier molecule at a time therethrough.

29. The method according to any one of claims 24 to 28, wherein the nanopore is located at the tip of a nanopipette.

30. The method according to any one of claims 23 to 29, wherein the method is for quantifying the amount of target analyte present in the sample, the method further comprising:
contacting the sample with a known concentration of carrier molecule; and
determining the ratio of occupied carrier molecules (i.e. carrier molecule-target analyte complexes) to unoccupied carrier molecules (i.e. carrier molecules to which no target analyte has bound).

31. The method according to claim 30, wherein determining the ratio of occupied carrier molecules to unoccupied carrier molecules comprises:
subjecting the sample to voltage-driven translocation through a nanopore; and
measuring the ion current signatures produced as the carrier molecules translocate through the nanopore,
wherein the ratio of single peaks to double peaks in the ion current signatures is indicative of the ratio of occupied carrier molecules to unoccupied carrier molecules.

32. A system for detecting and/or quantifying a target analyte in a sample, the system comprising:
a first electrolyte reservoir and a second electrolyte reservoir, the first and second reservoirs being separated by a barrier comprising a nanopore; and
optionally, electrodes for translocating molecules through the nanopore from the first electrolyte reservoir to the second electrolyte reservoir,
wherein at least one of the first and second electrolyte reservoirs comprises carrier molecules according to any one of claims 1 to 19.

33. The system according to claim 32, wherein the barrier comprises or consists of a membrane, optionally wherein the membrane is a biological membrane or a solid-state membrane.

34. A kit for the detection of a target analyte, the kit comprising:
a carrier molecule according to any one of claims 1 to 19; and
instructions for use.

35. The kit according to claim 34, wherein the kit further comprises a nanopore, optionally wherein the nanopore is comprised within a nanopipette.


SEQUENCE LISTING (3 sequences)

SEQ ID NO: 1 - DNA, 88 bp, Artificial sequence
  aptamer
aagcctttatttcaacggcaggaagacaaacacgatgggggggtatgatttgatgtggttgttgcatgatcgtggtctgtggtgctgt

SEQ ID NO: 2 - DNA, 68 bp, Artificial sequence
  aptamer
cgaaggggattcgaggggtgattgcgtgctccatttggtgttttttttttttgcaaggataaaaattt

SEQ ID NO: 3 - DNA, 87 bp, Artificial sequence
  aptamer
ctgaacaagaaaaatagcagaacttacgagccaggggaaacagtaaggcctaattaggtaaaggagtaagtgctcgaacgcttcaga